(19) United States
(12) Patent Application Publication
(10) Pub. No.: US 2024/0334892 A1
(43) Pub. Date: Oct. 10, 2024

(54) NOVEL WHEAT CENH3 ALLELES

(71) Applicant: Syngenta Crop Protection AG, Basel (CH)

(72) Inventors: Jian LV, Beijing (CN); Kun YU, Beijing (CN); Juan WEI, Beijing (CN); Chunxia LIU, Beijing (CN); Hongju ZHOU, Beijing (CN); Timothy KELLIHER, Research Triangle Park, NC (US)

(73) Assignee: Syngenta Crop Protection AG, Basel (CH)

(21) Appl. No.: 18/426,902

(22) Filed: Jan. 30, 2024

Related U.S. Application Data
(62) Division of application No. 17/286,950, filed on Apr. 20, 2021, now Pat. No. 12,016,286.

(30) Foreign Application Priority Data
Oct. 12, 2018 (CN) 201980063899.2

(51) Int. Cl. A01H 6/46

(52) U.S. Cl. CPC A01H 6/4678

(57) ABSTRACT

The present invention relates to wheat plants comprising a mutation causing an alteration of the amino acid sequence in centromere histone H3 (CENH3), which have the biological activity of a haploid inducer. Further, the present invention provides methods of generating the wheat plants of the present invention and haploid and doubled haploid wheat plants obtainable by crossing the wheat plants of the present invention with wildtype wheat plants.

### FIELD OF THE INVENTION

The disclosure relates to the field of agriculture. In particular, the disclosure relates to CenH3 proteins and polynucleotides encoding them, methods for the production of haploid as well as subsequent doubled haploid plants, and plants and seeds derived thereof, particularly in wheat species.

### CLAIM FOR PRIORITY

This application claims priority under the Paris Convention to PCT/CN2018/110063, filed Oct. 12, 2018, which is incorporated herein in its entirety.

### SEQUENCE LISTING

This application is accompanied by a sequence listing entitled 81696WOPCT_ST25.txt, created Sep. 30, 2019, which is approximately 112 kilobytes in size. This sequence listing is incorporated herein by reference in its entirety. This sequence listing is submitted herewith via EFS-Web, and is in compliance with 37 C.F.R. § 1.824(a)(2)-(6) and (b).

### BACKGROUND

A high degree of heterozygosity in breeding material can make plant breeding and selection for beneficial traits a very time consuming process. Extensive population screening, even with the latest molecular breeding tools, is both laborious and costly. The creation of haploid plants followed by chemical or spontaneous genome doubling has proven to be an efficient way to solve the problem of high heterozygosity and accelerate the breeding process. Such technology is also referred to as doubled haploid production system. The use of the doubled haploid production system has allowed breeders to achieve homozygosity at all loci in a single generation via whole-genome duplication. This effectively obviates the need for selfing or backcrossing, where normally at least 7 generations of selfing or backcrossing would be needed to reduce the heterozygosity to an acceptable level.

Haploid plants can be generated according to different methodologies. For instance, haploid plants can be produced in some crops by using a method referred to as microspore culture. However, this method is costly, time-consuming, and does not work in all crops. In some crop species, (doubled) haploid plants can be obtained by parthenogenesis of the egg cell or by elimination of one of the parental genomes. However, such methods are not optimal as they only work in few selected crop species and yield rather low rates of (doubled) haploid plants.

WO2011/044132 discloses a method for producing haploid plants consisting of inactivating or altering or knocking out the centromere-specific H3 (CenH3) protein in a plant. In a first step, the method consists of eliminating or knocking down the endogenous CenH3 gene in plant. In a second step, an expression cassette encoding a mutated or altered CenH3 protein is introduced in the plant. The mutated or altered CenH3 protein is generated by fusing an, optionally GFP-tagged, H3.3 N-terminal domain to the endogenous CenH3 histone-fold domain. Such methodology is also known as “GFP-tailswap” or “tailswap” (also reviewed in Britt and Kuppu, Front Plant Sci. 2016; 7: 357). The crossing of the plant harboring such tailswap with a wildtype plant (i.e., having functional endogenous CenH3 protein without a tailswap), causes uniparental genome elimination, which in turn results in the production of a haploid plant. Some haploid induction, though less frequent, was also found with N-terminal addition of GFP to endogenous CenH3 (no tailswap). However, this methodology is not ideal as it laborious, time-consuming and requires generating a transgenic plant.

WO2014/110274 describes a method for producing haploid plants consisting of crossing a first plant expressing an endogenous CenH3 gene to a second plant referred to as a haploid inducer plant having a genome from at least two species, wherein a majority of the genome is from a first species and the genome comprises a heterologous genomic region from a second species, wherein the heterologous genomic region encodes a CenH3 polypeptide different from the CenH3 of the first species (also described in Maheshwari et al, PLoS Genet. 2015 Jan. 26; 11(1):e1004970)). However, this methodology is not optimal as it suffers from the same pitfall as above—it is laborious, time-consuming and requires generating a transgenic plant. Further, the method is associated with low yield of haploid plants.

Other methods consist of introducing one or more point mutations leading to single amino acid change in the C-terminal histone fold domain of CenH3 protein or CenH3 gene coding the CenH3 protein. Examples of such mutations in the C-terminal histone fold domain of the CenH3 protein were reported in Karimi-Ashtiyani et al (2015) Proc Natl Acad Sci USA. 2015 Sep. 8; 112(36):11211-16; Kuppu, et al. (2015) PLoS Genet. 2015 Sep. 9; 11(9):e1005494. However, the success of such methods is mitigated as some, as not all of these mutations were found to be sufficient to induce uniparental genome elimination after crossing with a wildtype plant to produce a haploid plant.

Wheat (Triticum aestivum) is a particularly complex organism for editing or mutating its genes, as it is a hexaploid organism. Evolved over thousands of years and several cross-breedings with ancestor wheat species, Triticum aestivum comprises three genomes: A (possibly from T. monoccum or Einkorn wheat), B (possibly from T. searsii), and D (possibly from T. tauschii). Each genome has 7 chromosomes. Triticum aestivum has two copies of each genome, i.e., AA BB DD; thus it has 42 chromosomes total (6 complete genomes each with 7 chromosomes). See generally The Evolution of Wheat at www.cerealsdb.uk.net/cerealgenomics/WheatBP/Documents/DOC_Evolution.php, last accessed 10 Jul. 2019. Furthermore, an edit or mutation in one copy of one gene may not present observable effects in Triticum aestivum, as the additional 5 copies likely would compensate for the mutant copy. In order to truly observe a knockout mutation's effect, one would have to mutate all 6 copies.

Therefore, it remains elusive which mutation(s) or modification(s) in the CenH3 protein or CenH3 gene coding for the CenH3 protein are capable or sufficient to induce uniparental genome elimination to produce haploid plants. Thus, there remains a need in the art for alternative or improved methods that allow efficient generation of haploid plants (e.g. less labor-intensive, less-time consuming, less expensive, and/or do not necessarily require making a transgenic plant), which can subsequently be doubled to produce doubled haploid plants. With doubled haploid production systems, homozygosity may be achieved in one generation.

### SUMMARY

To meet this need, one embodiment of the invention is a wheat plant comprising at least an A genome, a B genome, and a D genome, wherein the B genome comprises a knock-out mutation in a CENH3 gene, and optionally wherein the D genome comprises a knock-out mutation in a CENH3 gene, and further wherein the A genome comprises a mutated CENH3 gene comprising at least one knock-down mutation at a 5′ splice site of an intron. In one aspect, the knock-down mutation is a restored frame shift mutation or a large deletion mutation. In another embodiment, the wheat plant is homozygous for a knock-out mutation in a CENH3 gene in the B genome. In an alternate embodiment, the wheat plant is biallelic for a knock-out mutation in a CENH3 gene in the B genome. In another embodiment, the wheat plant is homozygous for a knock-out mutation in a CENH3 gene in the D genome. In an alternate embodiment, the wheat plant is biallelic for a knock-out mutation in a CENH3 gene in the D genome. In yet another embodiment, the wheat plant is homozygous, biallelic, or a combination thereof for a knock-out mutation in a CENH3 gene in the B genome and the D genome. In another embodiment, the wheat plant is homozygous for the restored frame shift CENH3 mutation; or it is heterozygous for the restored frame shift CENH3 mutation; or it is biallelic for the restored frame shift CENH3 mutation.

Another aspect of the invention is a method of generating a haploid-inducing wheat plant, the method comprising: (a) obtaining at least a wheat plant cell comprising at least three genomes; (b) mutating two of the three genomes to obtain homozygous knock-out mutations in a CENH3 gene; (c) mutating the third genome to obtain a homozygous knock-down mutation in a CENH3 gene; and (d) generating a wheat plant therefrom comprising homozygous knock-out mutations in a CENH3 gene of two of the three genomes and further comprising a homozygous knock-down mutation in a CENH3 gene of the third genome; whereby the wheat plant generated from step (d) produces haploid progeny when crossed with a wildtype wheat plant. In one embodiment, the three genomes comprise an A genome, a B genome, and a D genome. In another, the knock-out mutations in a CENH3 gene occur in the B and D genomes. In yet another, the knock-down mutation in a CENH3 gene occurs in the A genome. In one aspect, the knock-down mutations in a CENH3 gene in the A genome are restored frame shift mutations. In another aspect, the restored frame shift mutations are selected from the group consisting of SEQ ID NO: 56, a nucleic acid sequence 70% identical to SEQ ID NO: 56, SEQ ID NO: 63, a nucleic acid sequence 70% identical to SEQ ID NO: 63, SEQ ID NO: 69, and a nucleic acid sequence 70% identical to SEQ ID NO: 69.

Another aspect of the invention is a wheat plant comprising a mutated CENH3 gene comprising at least one deletion mutation in the N-terminal domain resulting in a frame shift, a restored frame shift, or a large deletion. Yet another aspect is a wheat plant comprising a mutated CENH3 gene comprising at least one insertion mutation in the N-terminal domain resulting in a frame shift, a restored frame shift, or a large deletion.

Another aspect of the invention is a method of generating an engineered restored frame shift in a gene of a cell, comprising: (a) contacting the genome with a site-directed nuclease (“SDN”) and at least two guide nucleic acids, wherein the at least two guide nucleic acids target at least two target sequences within the gene; (b) permitting the SDN to cut the gene at the at least two target sequences, thereby losing an intervening sequence between the at least two target sequences; and allowing endogenous DNA repairs to occur; whereby the endogenous DNA repairs results in a gene having an engineered restored frame shift. In one embodiment, the lost intervening sequence of step (b) comprises (N) base pairs, where (N) is a multiple of 3.

Yet another aspect of the invention is a method of generating a haploid wheat plant, comprising: (a) obtaining a wheat plant; (b) crossing the wheat plant to the wheat plant comprising a mutated CENH3 gene; and (c) selecting a progeny generated from the crossing step; wherein the progeny is a haploid wheat plant. In one embodiment, the wheat plant of step (a) is the paternal parent. In another embodiment, the wheat plant of step (a) is the maternal parent. In another embodiment, the method comprises a further step of converting the progeny wheat plant into a doubled haploid wheat plant.

It is another aspect of the invention to provide a wheat plant comprising a mutated CENH3 allele comprising a nucleic acid sequence at least 70% identical to a sequence selected from the group consisting of SEQ ID NO: 53-73, wherein the mutation is an restored frame shift mutation, and wherein the wheat plant generates haploid progeny when crossed with a wildtype diploid wheat plant. In one embodiment, the wheat plant comprises at least one copy of the mutated CENH3 allele; in another embodiment, the wheat plant comprises at least two copies of the mutated CENH3 allele; in yet another embodiment, the wheat plant comprises at least three copies of the mutated CENH3 allele. In one embodiment, the mutated CENH3 allele comprises a nucleic acid sequence 80, 90, 95, or 100% identical to SEQ ID NO: 53-73.

TABLE 1
BRIEF DESCRIPTION OF THE SEQUENCES IN THE SEQUENCE LISTING

| SEQ ID NO: | Pimer ID (or Probe ID) | Location or function | Brief Description |
|---|---|---|---|
| 1 | S2-α1A | 5′ UTR of TaCenH3α-A | TaCenH3α-A genomic sequence cloning in |
| 2 | As-α1A/B/D | Exon 5 of TaCenH3α-A/B/D | Fielder |
| 3 | S-α1A/B/D | Exon 5 of TaCenH3α-A/B/D | |
| 4 | As-α1A | Exon 7 of TaCenH3α-A | |
| 5 | S-α1B | 5′ UTR of TaCenH3α-B | TaCenH3α-B genomic sequence cloning in |
| 6 | As-α1A/B | Exon 5 of TaCenHBα-A/B | Fielder |
| 7 | S-α1A/B/D | Exon 5 of TaCenH3α-A/B/D | |
| 8 | As-α1B/D | Exon 7 of TaCenH3α-B/D | |
| 9 | S2-α1D | 5′ UTR of TaCenH3α-D | TaCenH3α-D genomic sequence cloning in |
| 10 | As-α1A/B/D | Exon 5 of TaCenH3α-A/B/D | Fielder |
| 11 | S-α1A/B/D | Exon 5 of TaCenH3α-A/B/D | |
| 12 | As-α1B/D | Exon 7 of TaCenH3α-B/D | |
| 13 | qRT(A)-S | Sense primer | TaCenH3α-A expression |
| 14 | qRT(A)-As | Antisense primer | |
| 15 | qRT(A)-probe | Probe | |
| 16 | qRT(B)-S | Sense primer | TaCenH3α-B expression |
| 17 | qRT(B)-As | Antisense primer | |
| 18 | qRT(B)-probe | Probe | |
| 19 | qRT(B)-S | Sense primer | TaCenH3α-D expression |
| 20 | qRT(B)-As | Antisense primer | |
| 21 | qRT(B)-probe | Probe | |
| 22 | TQ1115-S | Sense primer | Control assay for the qRT-PCR, |
| | | | targeting ADP-ribosylation factor |
| 23 | TQ1115-As | Antisense primer | |
| 24 | TQ1115-probe | Probe | |
| 25 | gRNA1 | sgRNA targeting exon2-intron2 junction | |
| 26 | gRNA2 | sgRNA targeting intron3-exon4 junction | TaCenH3α sgRNAs |
| 27 | gRNA3 | sgRNA targeting exon1 | |
| 28 | gRNA4 | sgRNA targeting intron2-exon3 junction | |
| 29 | KW2917R | Antisense primer | |
| 30 | KW2917F1 | Sense primer | KASP assay - 2917 |
| 31 | KW2917F2 | Sense primer | |
| 32 | KWI1728R | Antisense primer | |
| 33 | KW11728F1 | Sense primer | KASP assay - 11728 |
| 34 | KW11728F2 | Sense primer | |
| 35 | KW11091R | Antisense primer | |
| 36 | KW11091F1 | Sense primer | KASP assay - 11091 |
| 37 | KW11091F2 | Sense primer | |
| 38 | KW11511R | Antisense primer | |
| 39 | KW11511F1 | Sense primer | RASP assay - 11511 |
| 40 | KW11511F2 | Sense primer | |
| 41 | KW11129R | Antisense primer | |
| 42 | KW11129F1 | Sense primer | KASP assay - 11129 |
| 43 | KW11129F2 | Sense primer | |
| 44 | e35S -S | Sense primer | |
| 45 | e35S -As | Antisense primer | Transgenic copy number check |
| 46 | e35S-probe | Probe | |
| 47 | PMI - S | Sense primer | |
| 48 | PMI - As | Antisense primer | Transgenic copy number check |
| 49 | PMI - probe | Probe | |
| 50 | FA | Sense primer | TaCENH3α-A edit sequencing primers |
| 51 | R3 | Antisense primer | |
| 52 | M13R | Antisense primer | Clone sequence primer |
| 53 | M13F | Sense primer | |
| 54 | F1 | Sense primer | TaCENH3α RT-PCR primer |
| 55 | R1 | Antisense primer | |
| 56 | A* genomic sequence in A004A | gRNA1, ins A; gRNA2, ins A | Genomic sequence in TaCENH3α-A |
| 57 | A* CDS sequence in A004A | Restored frame shift in N terminal | Restored frame shift in N terminal in |
| | | | TaCENH3α-A |
| 58 | a CDS sequence in A004A | Premature stop | Loss of function in TaCENH3α-A |
| 59 | A* protein sequence in A004A | Restored frame shift in N terminal | Restored frame shift in N terminal in |
| | | | TaCENH3α-A |
| 60 | a protein sequence in A004A | Premature stop | Loss of function in TaCENH3α-A |
| 61 | B genomic sequence in A004A | gRNA1, WT; gRNA2, WT | WT TaCENH3α-B |
| 62 | d genomic sequence in A004A | gRNA1, WT; gRNA2, \hat{ }A | Loss of function in TaCENH3α-D |
| 63 | A* genome sequence in C003A | gRNA1, ΔG; gRNA2 \hat{ }A | Transgenic copy number check |
| 64 | A* CDS sequence in C003A | gRNA1, ΔG; gRNA2, \hat{ }A | Restored frame shift in N terminal in |
| | | | TaCENH3α-A |
| 65 | A* protein sequence in C003A | gRNA1, ΔG; gRNA2, \hat{ }A | Restored frame shift in N terminal in |
| | | | TaCENH3α-A |
| 66 | b genomic sequence in C003A | gRNA1, WT; gRNA2, \hat{ }A | Loss of function in TaCSENH3α-B |
| 67 | b protein sequence in C003A | gRNA1, WT; gRNA2, \hat{ }A | Loss of function in TaCENH3α-B |
| 68 | d genomic sequence in C003A | gRNA1, WT; gRNA2, \hat{ }A | Loss of function in TaCENH3α-D |
| 69 | A* genomic sequence in A073A | gRNA3, \hat{ }A; gRNA4, ΔG | Restored frame- shift in N terminal in |
| | | | TaCENH3α-A |
| 70 | A* CDS sequence in A073A | gRNA3, \hat{ }A; gRNA4, ΔG | Restored frame shift in N terminal in |
| | | | TaCENH3α-A |
| 71 | A* protein sequence in A073A | gRKA3, \hat{ }A; gRNA4, ΔG | Restored frame shift in N terminal in |
| | | | TaCENH3α-A |
| 72 | b genomic sequence in A073A | gRNA3, WT; gRNA4, \hat{ }A | Loss of function in TaCENH3α-B |
| 73 | d genomic sequence in A073A | gRNA3, ΔGTC; gRNA4, \hat{ }A | Loss of function in TaCENH3α-D |
| 74 | | | Construct 24195 |
| 75 | | | Construct 24194 |
| 76 | | | Amino acid sequence lost in RES |
| 77 | | | Amino acid sequence added in RES |
| 78 | | | Amino acid sequence added in RES |
| 79 | SQ-1 primer | | |

### BRIEF DESCRIPTION OF THE FIGURES

FIG. 1 shows the TaCenH3α gene structure and relative gRNA locations. Exons are numbered and represented by thick bars. Introns are represented by thin lines. Length of both is represented by width.

### DEFINITIONS

This invention is not limited to the particular methodology, protocols, cell lines, plant species or genera, constructs, and reagents described herein. The terminology used herein is for the purpose of describing particular embodiments only, and is not intended to limit the scope of the present invention, which will be limited only by the appended claims. It must be noted that as used herein and in the appended claims, the singular forms “a,” “and,” and “the” include plural reference unless the context clearly dictates otherwise. Thus, for example, reference to “a plant” is a reference to one or more plants and includes equivalents thereof known to those skilled in the art, and so forth. As used herein, the word “or” means any one member of a particular list and also includes any combination of members of that list (i.e., includes also “and”).

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means±1° C., preferably ±0.5° C. Where the term “about” is used in the context of this invention (e.g., in combinations with temperature or molecular weight values) the exact value (i.e., without “about”) is preferred.

As used herein, the term “amplified” means the construction of multiple copies of a nucleic acid molecule or multiple copies complementary to the nucleic acid molecule using at least one of the nucleic acid molecules as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, PERSING et al., Ed., American Society for Microbiology, Washington, D.C. (1993). The product of amplification is termed an “amplicon.”

The term “biallelic” refers to a gene pair that is neither homozygous (AA or aa) nor heterozygous (Aa). Rather, both genes in the pair have been edited but not identically. For example, the CenH3 gene pair on the A chromosome in this invention may comprise one RFS mutation in one allele resulting in a knock-down of the gene upon expression, while the other allele comprises a knock-out mutation. This may be indicated symbolically as “A*a” and is indicative of a biallelic mutation.

The term “specific DNA sequence” indicates a polynucleotide sequence having a nucleotide sequence homology of more than 80%, preferably more than 85%, more preferably more than 90%, even more preferably more than 95%, still more preferably more than 97%, most preferably more than 99% with another named sequence.

“cDNA” refers to a single-stranded or a double-stranded DNA that is complementary to and derived from mRNA. The term “centromere-specific variant of histone H3 protein” (“CenH3 protein” or simply “CENH3”), as used herein, refers to a protein that is a member of the kinetochore complex. CenH3 protein is also known as CENP-A protein. The kinetochore complex is located on chromatids where the spindle fibers attach during cell division to pull sister chromatids apart. CenH3 proteins belong to a well-characterized class of proteins that are variants of H3 histone proteins. These proteins are essential for proper formation and function of the kinetochore, and help the kinetochore associate with DNA. Cells that are deficient in CenH3 fail to localize kinetochore proteins on chromatids and show strong chromosome segregation defects (i.e., all chromosomes from the plant expressing the deficient CenH3 protein are eliminated or lost, leading to a change in the ploidy of somatic cells (e.g., reduction in the number of chromosome set such as diploid to haploid)). Therefore, CenH3 proteins have been subject to intensive research for their potential use in doubled haploid production system. CenH3 proteins are characterized by a variable tail domain (also referred to as “N-terminal domain” or “N-terminal tail domain”) and a conserved histone fold domain (also referred to as “C-terminal domain”) made up of three alpha-helical regions connected by loop sections. The CenH3 histone fold domain is relatively well conserved between CenH3 proteins from different species. The histone fold domain is located at the carboxyl terminus of an endogenous CenH3 protein. In contrast to the histone-fold domain, the N-terminal tail domain of CenH3 is highly variable even between closely related species.

“CenH3-encoding polynucleotide having one or more active mutations” refers to a non-endogenous or endogenous mutated CenH3-encoding polynucleotide that encodes a CenH3 protein having one or more active mutations, which when present in a plant in the absence of its endogenous CenH3-encoding polynucleotide and/or endogenous CenH3 protein, allows the plant to be viable, and allows generation of haploid progeny, or progeny with aberrant ploidy, when the plant is crossed with a wild-type plant. The plant comprising a CenH3-encoding polynucleotide having one or more active mutations may be referred to as a “modified plant.” The percentage of haploid progeny or progeny with aberrant ploidy that is generated upon crossing with a wild-type plant can, for instance, be at least 0.1, 0.5, 1, 5, 10, 20 percent or more. A mutation that causes a transition from the endogenous CenH3-encoding polynucleotide to a CenH3-encoding polynucleotide having one or more active mutations is herein referred to as an active mutation. An active mutation in a CenH3 protein context may result, among other things, in reduced centromere loading, a less functional CenH3 protein and/or a reduced functionality in the separation of chromosomes during cell division. One or more active mutations may be introduced into the CenH3-encoding polynucleotide by any of several methods well-known to the skilled person, for example, by random mutagenesis, such as induced by treatment of seeds or plant cells with chemicals or radiation, targeted mutagenesis, the application of endonucleases, by generation of partial or complete protein domain deletions, or by fusion with heterologous sequences.

A plant may be made to lack the endogenous CenH3-encoding polynucleotide by knocking out or inactivating the endogenous CenH3-encoding polynucleotide. Alternatively, the endogenous CenH3-encoding polynucleotide may be modified to encode an inactive or non-functional CenH3 protein.

The modified plant comprising the CenH3-encoding polynucleotide having one or more active mutations as taught herein may be crossed to a wild-type plant either as a pollen parent or as an ovule parent. In an embodiment, a CenH3 protein having one or more active mutations may comprise 1, 2, 3, 4, 5, 6, 7, 8, 9, 10, 12, 15, 20 or more amino acid changes relative to the endogenous CenH3 protein. In an embodiment, a CenH3-encoding polynucleotide having one or more active mutations has 70, 75, 80, 85, 90, 95, 96, 97, 98, 99, 99.5 percent sequence identity to the endogenous CenH3-encoding polynucleotide, preferably over the full length. The skilled person would readily be able to ascertain whether or not a modified plant as taught herein comprises one or more active mutations. For example, the skilled person may make use of predictive tools such as SIFT (Kumar P, Henikoff S, Ng PC. (2009) Predicting the effects of coding non-synonymous variants on protein function using the SIFT algorithm. Nat Protoc; 4(7): 1073-81. doi: 10.1038/nprot.2009.86) to propose such active mutation. The one or more active mutations may then be made in a plant, and expression of endogenous CenH3 protein in the plant should be knocked out. The plant may be considered to comprise one or more active mutations when the percentage of haploid progeny or progeny with aberrant ploidy that is generated upon crossing with a wild-type plant is at least 0.1, 0.5, 1, 5, 10, 20 percent or more.

Crossing a plant that lacks an endogenous CenH3-encoding polynucleotide, or that lacks expression of endogenous CenH3 protein, and that expresses a CenH3 protein having one or more active mutations either as a pollen or as an ovule parent with a wildtype plant (i.e., it expresses an endogenous CenH3 protein) results in progeny that is haploid or shows aberrant ploidy. Such a plant comprises only chromosomes of the parent that expresses the endogenous CenH3 protein, and no chromosomes of the plant expressing the CenH3 protein having one or more active mutation.

The term “aberrant ploidy” as used herein refers to a situation where a cell comprises an aberrant or abnormal number of sets of chromosomes. For instance, a cell having one or three sets of chromosomes per cell when the usual number is two is a cell having aberrant ploidy. In the present invention, the active mutant CenH3 proteins and methods using them, can be used to generate mutant plants having aberrant ploidy, e.g., to generate haploid plants while the non-mutant plant is diploid. The haploid plants can be used to accelerate breeding programs to create homozygous lines and obviate the need for inbreeding.

The term “chimeric construct”, “chimeric gene”, “chimeric polynucleotide” or chimeric nucleic acid” (and similar terms) as used herein refers to a construct or molecule comprising two or more polynucleotides of different origin assembled into a single nucleic acid molecule. The term “chimeric construct”, “chimeric gene”, “chimeric polynucleotide” or “chimeric nucleic acid” refers to any construct or molecule that contains (1) polynucleotides (e.g., DNA), including regulatory and coding polynucleotides that are not found together in nature (i.e., at least one of polynucleotides is heterologous with respect to at least one of its other polynucleotides), or (2) polynucleotides encoding parts of proteins not naturally adjoined, or (3) parts of promoters that are not naturally adjoined. Further, a chimeric construct, chimeric gene, chimeric polynucleotide or chimeric nucleic acid may comprise regulatory polynucleotides and coding polynucleotides that are derived from different sources, or comprise regulatory polynucleotides and coding polynucleotides derived from the same source, but arranged in a manner different from that found in nature. In a preferred aspect of the present invention the chimeric construct, chimeric gene, chimeric polynucleotide or chimeric nucleic acid comprises an expression cassette comprising a polynucleotides of the present invention under the control of regulatory polynucleotides, particularly under the control of regulatory polynucleotides functional in plants.

The term “chromosome” is used herein as recognized in the art as meaning the self-replicating genetic structure in the cellular nucleus containing the cellular DNA and bearing the linear array of genes.

A “coding polynucleotide” is a polynucleotide that is transcribed into RNA, such as mRNA, rRNA, tRNA, snRNA, sense RNA or antisense RNA. Preferably the RNA is then translated in an organism to produce a protein. It may constitute an “uninterrupted coding polynucleotide”, i.e., lacking an intron, such as in a cDNA, or it may include one or more introns bounded by appropriate splice junctions. An “intron” is a poly(ribo)nucleotide which is contained in the primary transcript but which is removed through cleavage and religation of the RNA within the cell to create the mature mRNA that can be translated into a protein.

The term “doubled haploid plant” as used herein refers to a genotype formed when haploid cells undergo chromosome doubling. Artificial production of doubled haploids is important in plant breeding. Doubled haploids can be produced in vivo or in vitro. Haploid embryos are produced in vivo by parthenogenesis, pseudogamy, or chromosome elimination. A wide variety of in vitro methods are known for generating doubled haploid organisms from haploid organisms. A non-limiting example of a method for generating doubled haploid in vitro consist of treating somatic haploid cells, haploid embryos, haploid seeds, or haploid plants produced from haploid seeds with a chromosome doubling agent such as colchicine. In the present invention, homozygous double haploid plants can be regenerated from haploid cells by contacting the haploid cells with chromosome doubling agents, such as colchicine, anti-microtubule herbicides, or nitrous oxide to create homozygous doubled haploid cells. Methods of chromosome doubling are disclosed in, for example, U.S. Pat. No. 5,770,788; 7,135,615, and US Patent Publication Nos. 2004/0210959 and 2005/0289673; Antoine-Michard, S. et al., Plant Cell, Tissue Organ Cult., Cordrecht, the Netherlands, Kluwer Academic Publishers 48(3):203-207 (1997); Kato, A., Maize Genetics Cooperation Newsletter 1997, 36-37; and Wan, Y. et al., Trends Genetics 77: 889-892 (1989). Wan, Y. et al., Trends Genetics 81: 205-21 1 (1991), the disclosures of which are incorporated herein by reference. Double haploid plants can be further crossed to other plants to generate F1, F2, or subsequent generations of plants with desired traits. Conventional inbreeding procedures take seven generations to achieve approximately complete homozygosity, whereas doubled haploidy achieves it in one generation.

The term “E0” refers to the edited plant in the first instance. That is, a plant cell which is edited by, e.g., CRISPR, and then allowed to mature into a plant has become the E0 plant. An E1 plant is the edit-comprising progeny (usually but not necessarily self-fertilized) of the E0. Likewise, an E2 plant is the edit-comprising progeny (usually but not necessarily self-fertilized) of the E1 plant. An E3, E4, E5, etc., plant is likewise generationally removed from the E0 plant.

The terms “gene editing,” “editing,” “genome editing,” “GE,” and the like refer to site-specific mutations made at a target sequence. This may also be referred to as “targeted mutagenesis.” As used herein, the term “targeted mutagenesis” or “mutagenesis strategy” refers to any method of mutagenesis that results in the intentional mutagenesis of a chosen gene. Targeted mutagenesis includes the methods CRISPR, TILLING, TALEN, and other methods not yet discovered but which may be used to achieve the same outcome. Mutagenesis may be performed in accordance with any of the techniques known in the art, such as, and not limited to, synthesizing an oligonucleotide having one or more mutations within the sequence of a particular regulatory sequence. In particular, site-specific mutagenesis is a technique useful in the preparation of promoter mutants, through specific mutagenesis of the underlying DNA. RNA-guided endonucleases (“RGEN,” e.g., CRISPR/Cas9) may also be used. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered. See generally, U.S. Pat. No. 10,285,348, incorporated by reference herein in its entirety.

The terms “edited N-terminal tail” or “edited N-terminal domain” are used interchangeably here throughout.

The term “endogenous” as used in the context of the present invention in combination with protein or gene means that said protein or gene originates from the plant in which it is still contained. Often an endogenous gene will be present in its normal genetic context in the plant. In another context, the term “endogenous” can refer to normal functions of a cell. For example and not by way of limitation, “endogenous DNA repair” refers to a cell's normal DNA repair mechanisms, enzymes, and processes.

The term “expression” when used with reference to a polynucleotide, such as a gene, ORF or portion thereof, or a transgene in plants, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (e.g. if a gene encodes a protein), through “translation” of mRNA.

Gene expression can be regulated at many stages in the process. For example, in the case of antisense or dsRNA constructs, respectively, expression may refer to the transcription of the antisense RNA only or the dsRNA only. In embodiments, “expression” refers to the transcription and stable accumulation of sense (mRNA) or functional RNA. “Expression” may also refer to the production of protein.

The terms “homology”, “sequence similarity” or “sequence identity” of nucleotide or amino acid sequences mean a degree of identity or similarity of two or more sequences and may be determined conventionally by using known software or computer programs such as the Best-Fit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2:482-489 (1981), to find the best segment of identity or similarity between two sequences. Sequence comparison between two or more polynucleotides or polypeptides is generally performed by comparing portions of the two sequences over a comparison window to identify and compare local regions of sequence similarity. The comparison window is generally from about 20 to 200 contiguous nucleotides. Gap performs global alignments: all of one sequence with all of another similar sequence using the method of Needleman and Wunsch, J. Mol. Biol. 48:443-453 (1970). When using a sequence alignment program such as BestFit to determine the degree of DNA sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

The term “locus” refers to a position (e.g., of a gene, a genetic marker, or the like) on a chromosome of a given species.

The term “primer”, as used herein, refers to an oligonucleotide which is capable of annealing to the amplification target allowing a DNA polymerase to attach, thereby serving as a point of initiation of DNA synthesis when placed under conditions in which synthesis of primer extension product is induced, e.g., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer is generally sufficiently long to prime the synthesis of extension products in the presence of the agent for polymerization. The exact lengths of the primers will depend on many factors, including temperature and composition (A/T and G/C content) of primer. A pair of bi-directional primers consists of one forward and one reverse primer as commonly used in the art of DNA amplification such as in PCR amplification. It will be understood that “primer,” as used herein, may refer to more than one primer, particularly in the case where there is some ambiguity in the information regarding the terminal sequence(s) of the target region to be amplified. Hence, a “primer” includes a collection of primer oligonucleotides containing sequences representing the possible variations in the sequence or includes nucleotides which allow a typical base pairing. The oligonucleotide primers may be prepared by any suitable method. Methods for preparing oligonucleotides of specific sequence are known in the art, and include, for example, cloning and restriction of appropriate sequences, and direct chemical synthesis. Chemical synthesis methods may include, for example, the phospho di or tri-ester method, the diethylphosphoramidate method and the solid support method disclosed in, for example, U.S. Pat. No. 4,458,066. The primers may be labeled, if desired, by incorporating means detectable by, for instance, spectroscopic, fluorescence, photochemical, biochemical, immunochemical, or chemical means. Template-dependent extension of the oligonucleotide primer(s) is catalyzed by a polymerizing agent in the presence of adequate amounts of the four deoxyribonucleotide triphosphates (dATP, dGTP, dCTP and dTTP, i.e. dNTPs) or analogues, in a reaction medium which is comprised of the appropriate salts, metal cations, and pH buffering system. Suitable polymerizing agents are enzymes known to catalyze primer- and template-dependent DNA synthesis. Known DNA polymerases include, for example, E. coli DNA polymerase I or its Klenow fragment, T4 DNA polymerase, and Taq DNA polymerase. The reaction conditions for catalyzing DNA synthesis with these DNA polymerases are known in the art. The products of the synthesis are duplex molecules consisting of the template strands and the primer extension strands, which include the target sequence. These products, in turn, serve as template for another round of replication. In the second round of replication, the primer extension strand of the first cycle is annealed with its complementary primer; synthesis yields a “short” product which is bound on both the 5′- and the 3′-ends by primer sequences or their complements. Repeated cycles of denaturation, primer annealing, and extension result in the exponential accumulation of the target region defined by the primers. Sufficient cycles are run to achieve the desired amount of polynucleotide containing the target region of nucleic acid. The desired amount may vary, and is determined by the function which the product polynucleotide is to serve. The PCR method is well described in handbooks and known to the skilled person. After amplification by PCR, the target polynucleotides may be detected by hybridization with a probe polynucleotide which forms a stable hybrid with that of the target sequence under low, moderate or even highly stringent hybridization and wash conditions. If it is expected that the probes will be essentially completely complementary (i.e., about 99% or greater) to the target sequence, highly stringent conditions may be used. If some mismatching is expected, for example if variant strains are expected with the result that the probe will not be completely complementary, the stringency of hybridization may be lessened. However, conditions are typically chosen which rule out nonspecific/adventitious binding. Conditions, which affect hybridization, and which select against nonspecific binding are known in the art, and are described in, for example, Sambrook and Russell, 2001. Generally, lower salt concentration and higher temperature increase the stringency of hybridization conditions. “PCR primer” is preferably understood within the scope of the present invention to refer to relatively short fragments of single-stranded DNA used in the PCR amplification of specific regions of DNA.

The terms “protein,” “peptide” and “polypeptide” are used interchangeably herein.

The term “promoter” refers to a polynucleotide, usually upstream (5′) of its coding polynucleotide, which controls the expression of the coding polynucleotide by providing the recognition for RNA polymerase and other factors required for proper transcription.

The term “site-directed nuclease” refers to any enzyme guided by a nucleotide sequence to a target sequence within a strand of DNA. The site-directed nuclease is preferably CRISPR-based, but could also be a meganuclease, a transcription-activator like effector nuclease (TALEN), or a zinc finger nuclease. Site-directed nuclease(s) may be referred to by the acronym “SDN.” SDNs include but are not limited to meganucleases (MNs), zinc-finger nucleases (ZFNs), transcription-activator like effector nucleases (TALENs), Cas9 nuclease, Cpf1 (Cas12a) nuclease, dCas9-FokI, dCpf1-FokI, chimeric Cas9-cytidine deaminase, chimeric Cas9-adenine deaminase, chimeric FEN1-FokI, and Mega-TALs, a nickase Cas9 (nCas9), chimeric dCas9 non-FokI nuclease and dCpf1 non-FokI nuclease; and further wherein the guide nucleic acid is a guide RNA.

The terms “stringent conditions” or “stringent hybridization conditions” include reference to conditions under which a polynucleotide will hybridize to its target sequence to a detectably greater degree than other sequences (e.g., at least 2-fold over background). Stringent conditions are sequence-dependent and will be different in different circumstances. By controlling the stringency of the hybridization and/or washing conditions, target polynucleotides can be identified which are 100% complementary to the probe (homologous probing). Alternatively, stringency conditions can be adjusted to allow some mismatching in sequences so that lower degrees of similarity are detected (heterologous probing). Typically, stringent conditions will be those in which the salt concentration is less than approximately 1.5 M Na ion, typically about 0.01 to 1.0 M Na ion (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions also may be achieved with the addition of destabilizing agents such as formamide. Exemplary low stringency conditions include hybridization with a buffer solution of 30 to 35% formamide, 1 M NaCl, 1% SDS (w/v; sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Specificity is typically the function of post-hybridization washes, the critical factors being the ionic strength and temperature of the final wash solution. For DNA-DNA hybrids, the Tm can be approximated from the equation of Meinkoth and Wahl (Anal. Biochem., 138:267-284, 1984): Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with approximately 90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology Hybridization with Nucleic Acid Probes, Part I, Chapter 2 “Overview of principles of hybridization and the strategy of nucleic acid probe assays”, Elsevier, N.Y. (1993); and Current Protocols in Molecular Biology, Chapter 2, Ausubel, et al., eds., Greene Publishing and Wiley-Interscience, New York (1995). Methods of stringent hybridization are known in the art which conditions can be calculated by means known in the art. This is disclosed in Sambrook et al., Molecular Cloning: A Laboratory Manual, 2nd Ed., Cold Spring Harbor Laboratory Press, 1989, Cold Spring Harbor, N.Y. and Current Protocols in Molecular Biology, Ausebel et al, eds., John Wiley and Sons, Inc., 2000. Methods of determining percent sequence identity are known in the art, an example of which is the GCG computer sequence analysis software (GCG, Inc, Madison Wis.).

As used herein, the term “restored frame shift” (“RFS”) refers to a mutation or series of mutations in a gene which, individually or in combination, interrupts the coding sequence of a gene yet does not alter the frame of the coding sequence. This may also be referred to as “restoring frame synchronization.” For example, a DNA coding sequence comprises a series of codons. Each codon comprises three nucleotides, and each codon-when transcribed into RNA-codes for one amino acid upon translation. An insertion/deletion mutation (“indel”) of one or two nucleotides into the coding sequence will cause a shift in the coding frame (a “frame shift”). However, insertions or deletions, whether individually or in combination, which occur cumulatively as a multiple of three will restore the codons to its original frame, even if the coding sequence itself is altered. See, e.g., B. N. Ames and H. J. Whitfield, Jr., Frameshift Mutagenesis in Salmonella, COLD SPRING HARB. SYMP. QUANT. BIOL. 31:221-225 (1966). For example, and within the scope of this definition, a sequence comprising at least two indel mutation deletions-whether consecutive or not-and in which the indel mutations cause the reading frame to be restored to its original frame is a sequence comprising a restored frameshift mutation. The term “engineered restored frame shift” may also be used to describe a RFS mutation which has been created by genome editing or genome modification.

As used herein, the term “large deletion” (“LD”) refers to a mutation which causes the loss of several consecutive nucleotides. In particular, a large deletion refers to the loss of 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or 100 or more nucleotides. In some embodiments, the sequence lost in an LD will be a multiple of 3 (i.e., 3, 6, 9, 12, 15, 18, 21, 24, 27, 30, etc.) In other embodiments, an LD mutation may also occur in conjunction with an indel mutation elsewhere in the same sequence, thereby causing a restored frame shift mutation.

In the context of the present invention, the use of the term “wildtype” or “wildtype plant” refers to a plant which does not carry a mutant CenH3 protein or gene (i.e., does not comprise one or more active mutations taught here) and which endogenously expresses or produces functional CenH3 genes and proteins.

### DETAILED DESCRIPTION

Here, we induced alternative splicing in wheat (Triticum aestivum) by applying CRISPR-Cas9 to edit cis-splicing sequences including 5′ and 3′ splice sites. We chose wheat as the target model organism because wheat is hexaploid, which gives wheat functional genomic redundancy. As a target gene, we chose the centromeric protein-encoding gene CENTROMERIC HISTONE 3 (“CenH3”) because modifications in this gene should produce plants with value for crop breeding. CenH3 is responsible for the faithful segregation of chromosomes during cell division. Unlike H3 and other conventional histones, CENH3 has a long, hypervariable N-terminal tail. See J. Monen, et al. Separase Cleaves the N-Tail of the CENP-A Related Protein CPAR-1 at the Meiosis I Metaphase-Anaphase Transition in C. elegans, PLOS ONE 10:e0125382 (2015). Directed or natural modification of the tail triggers compensatory changes in the kinetochore, which may enable CENH3 to drive speciation through impairing meiosis or inhibiting zygotic chromosome segregation. See I. Lermontova, et al., Knockdown of CENH3 in Arabidopsis reduces mitotic divisions and causes sterility by disturbed meiotic chromosome segregation, PLANT J 68:40-50 (2011) and M. Ravi and R. Bondada, Genome Elimination by Tailswap CenH3: In Vivo Haploid Production in Arabidopsis thaliana, METHODS MOL BIOL 1469:77-99 (2016). Swapping the N-terminal tail with an H3 tail led to haploid induction in Arabidopsis (M. Ravi and S. Chan, Haploid plants produced by centromere-mediated genome elimination, NATURE 464:615-618 (2010)) and maize (T. Kelliher, et al., Maternal Haploids are Preferentially Induced by CENH3-tailswap Transgenic Complementation in Maize, FRONT. PLANT SCI., doi.org/10.3389/fpls.2016.00414 31(7):414 (2016). Haploid induction is an aberrant reproductive process that leads to ploidy reduction from one generation to the next. Haploids can be doubled to produce inbred lines, saving six generations of self-pollination normally required to generate new pure-bred stocks. Delivering the tail-swap approach to crops requires multiple generations to assemble the native allele knockout and stable insertion of transgenes. We were able to induce AS by directly editing N-terminal sequences in wheat CenH3. These novel CenH3 sequences were studied to determine whether and in what combination mutant CENH3 proteins might cause haploid induction in wheat. Under the circumstances we describe, it does.

Therefore, one embodiment of the invention is a wheat plant comprising at least an A genome, a B genome, and a D genome, wherein the B genome comprises a knock-out mutation in a CENH3 gene, and optionally wherein the D genome comprises a knock-out mutation in a CENH3 gene, and further wherein the A genome comprises a mutated CENH3 gene comprising at least one knock-down mutation at a 5′ splice site of an intron. In one aspect, the knock-down mutation is a restored frame shift mutation or a large deletion mutation. In another embodiment, the wheat plant is homozygous for a knock-out mutation in a CENH3 gene in the B genome. In an alternate embodiment, the wheat plant is biallelic for a knock-out mutation in a CENH3 gene in the B genome. In another embodiment, the wheat plant is homozygous for a knock-out mutation in a CENH3 gene in the D genome. In an alternate embodiment, the wheat plant is biallelic for a knock-out mutation in a CENH3 gene in the D genome. In yet another embodiment, the wheat plant is homozygous, biallelic, or a combination thereof for a knock-out mutation in a CENH3 gene in the B genome and the D genome. In another embodiment, the wheat plant is homozygous for the restored frame shift CENH3 mutation; or it is heterozygous for the restored frame shift CENH3 mutation; or it is biallelic for the restored frame shift CENH3 mutation.

Another aspect of the invention is a method of generating a haploid-inducing wheat plant, the method comprising: (a) obtaining at least a wheat plant cell comprising at least three genomes; (b) mutating two of the three genomes to obtain homozygous knock-out mutations in a CENH3 gene; (c) mutating the third genome to obtain a homozygous knock-down mutation in a CENH3 gene; and (d) generating a wheat plant therefrom comprising homozygous knock-out mutations in a CENH3 gene of two of the three genomes and further comprising a homozygous knock-down mutation in a CENH3 gene of the third genome; whereby the wheat plant generated from step (d) produces haploid progeny when crossed with a wildtype wheat plant. In one embodiment, the three genomes comprise an A genome, a B genome, and a D genome. In another, the knock-out mutations in a CENH3 gene occur in the B and D genomes. In yet another, the knock-down mutation in a CENH3 gene occurs in the A genome. In one aspect, the knock-down mutations in a CENH3 gene in the A genome are restored frame shift mutations. In another aspect, the restored frame shift mutations are selected from the group consisting of SEQ ID NO: 56, a nucleic acid sequence 70% identical to SEQ ID NO: 56, SEQ ID NO: 63, a nucleic acid sequence 70% identical to SEQ ID NO: 63, SEQ ID NO: 69, and a nucleic acid sequence 70% identical to SEQ ID NO: 69.

Another aspect of the invention is a wheat plant comprising a mutated CENH3 gene comprising at least one deletion mutation in the N-terminal domain resulting in a frame shift, a restored frame shift, or a large deletion. Yet another aspect is a wheat plant comprising a mutated CENH3 gene comprising at least one insertion mutation in the N-terminal domain resulting in a frame shift, a restored frame shift, or a large deletion.

Another aspect of the invention is a method of generating an engineered restored frame shift in a gene of a cell, comprising: (a) contacting the genome with a site-directed nuclease (“SDN”) and at least two guide nucleic acids, wherein the at least two guide nucleic acids target at least two target sequences within the gene; (b) permitting the SDN to cut the gene at the at least two target sequences, thereby losing an intervening sequence between the at least two target sequences; and allowing endogenous DNA repairs to occur; whereby the endogenous DNA repairs results in a gene having an engineered restored frame shift. In one embodiment, the lost intervening sequence of step (b) comprises (N) base pairs, where (N) is a multiple of 3.

Yet another aspect of the invention is a method of generating a haploid wheat plant, comprising: (a) obtaining a wheat plant; (b) crossing the wheat plant to the wheat plant comprising a mutated CENH3 gene; and (c) selecting a progeny generated from the crossing step; wherein the progeny is a haploid wheat plant. In one embodiment, the wheat plant of step (a) is the paternal parent. In another embodiment, the wheat plant of step (a) is the maternal parent. In another embodiment, the method comprises a further step of converting the progeny wheat plant into a doubled haploid wheat plant.

It is another aspect of the invention to provide a wheat plant comprising a mutated CENH3 allele comprising a nucleic acid sequence at least 70% identical to a sequence selected from the group consisting of SEQ ID NO: 56-73, wherein the mutation is an restored frame shift mutation, and wherein the wheat plant generates haploid progeny when crossed with a wildtype diploid wheat plant. In one embodiment, the wheat plant comprises at least one copy of the mutated CENH3 allele; in another embodiment, the wheat plant comprises at least two copies of the mutated CENH3 allele; in yet another embodiment, the wheat plant comprises at least three copies of the mutated CENH3 allele. In one embodiment, the mutated CENH3 allele comprises a nucleic acid sequence 80, 90, 95, or 100% identical to SEQ ID NO: 56-73.

### EXAMPLES

### Example 1: The Theory Behind Using Two N-Terminal Guide RNAs

CENH3-tailswap transgenes, when expressed heterologously in a line where the native CENH3 genes are knocked out, leads to haploid induction. See, e.g., U.S. Patent Application Publication No. 2019/0136250, incorporated herein by reference. This is called the tailswap approach. Importantly, there are no wildtype alleles in tailswap haploid inducer lines. The transgenes are inferred to have partial function and are capable of generating centromeres that are stable enough to get a normally-developing plant when homozygous. However, when the tailswap transgenes are heterologous with wildtype CENH3 in a cell, the tailswap transgenes are unstable and lead to successful haploid induction during outcross. It is critical in these designs of tailswap plants that the native CENH3 genes are knocked out and that the tailswap transgenes have significant alterations of the N-terminal domain combined with only minor, or preferably zero, alterations to the C-terminal domain. Haploid induction will not occur even if the mutant CENH3 genes encode CENH3 proteins that retain normal or near-normal functionality.

In order to achieve haploid induction in wheat, we directly edited the six CENH3α genes to knock out several copies and create modifications to the N-terminal domain (leaving the C-terminal domain intact) in still other copies. Based on our experiments measuring the gene expression of the A, B, & D genomes's CENH3α genes, we particularly focused on creating N-terminal modifications in the A genome, and knockouts in the B and D genome. If our edits were successful, we would leave zero copies of CENH3α normal (intact): All genes would be edited, but the outcomes of the editing would differ. Importantly, our editing design did not include any CENH3 transgenes-we simply wanted to create the partial function, N-terminal modified version of the A genome CENH3α through direct editing.

Typically, large alterations to regions encoding proteins can be achieved through CRISPR SDN II genome editing (also called allele replacement (“AR”) or homologous recombination (“HR”)), but the efficiency of that technology is extremely low in plants and only rarely achieved in wheat. Therefore, we designed an editing strategy using two guide RNAs (“gRNAs”) that had the potential to modify CENH3α to create a partial loss of function allele that had a large alteration (a change of more than 5 amino acids) of the N-terminal domain and a native (unaltered) sequence for the C-terminal domain. This would require specific cuts at both guide RNA sites. We knew that the selected guide RNAs would also edit the B and D genome's copies of CENH3α, and this was intentional. In fact, due to the unpredictability of each specific editing outcome for two guide RNAs, we expected that most edited alleles would be full loss of function alleles (in A, B and D genome copies), due to frame-shifts in the coding sequence that resulted in pre-mature stop codons that truncated the CENH3α protein product and thus were complete knockouts of the native gene. However we also knew that if all copies of the CENH3α gene were knocked out, the plants would die because partial function of CENH3α is required for plant development. If the same guides that created a modified, haploid inducer allele in the A genome could simultaneously (in the same plants) knock out the B and D genome's alleles, it would help us: The result would be a perfectly conceived haploid inducer line. Thus our aim was to use two gRNAs to mimic the tailswap transgenic system by direct editing, but our key inventive step was instead of doing any allele replacement or CENH3α transgene, we generated novel, modified variants through small indels created by the nuclease cutting at the two guide RNAs in the N-terminal domain.

However, not every pair of guides possible had the potential to combine to create an altered N-terminal domain paired with a functional C-terminal domain: Not every pair of guides could produce those “edited N-terminal tail” altered copies with partial functions and haploid induction potential. Many of the guides, after checking what the edits would lead to in terms of the amino acid sequence, would lead to premature stop codons. In other words, we realized that we had to specifically select guide RNAs that we predicted could generate a combination of edits at the target sites that would generate amino acid sequences in the mature CENH3α protein product that contained dramatic alterations to the N-terminal domain, but left the C-terminal domain unaffected. In particular, we planned to screen plants and identify those that contained such productive, N-terminal modified alleles for the A genome's copies of CENH3α, and which also had knock out alleles for the B and D genome's copies of CENH3α. Knowing that site-directed nuclease-mediated editing does not always occur right away during transformation, we reasoned that in the E0 generation, we may generate knockout (full loss of function) alleles for some of the copies and also some partial loss of function (haploid inducer) alleles for still other copies of the CENH3α gene, and that these materials may be able to generate haploids by self-pollination—assuming the male and female gametes (sperm and egg cells) have different edits or different combinations of alleles and thus potentially different centromeres. In other words, we thought we may find some haploids in the E1 generation plants. We hoped to let those observations guide us towards selecting certain E1 plants to genotype (i.e., genotype diploid siblings from populations that gave rise to some haploids upon self-pollination) and identify the E1 plants that had the partial loss of function alleles in a homozygous state. If we could do that then we would use those particular plants to test the haploid induction rate via outcrossing. In summary, we thought that once we created stably-mutated lines we would be able to test whether they were really haploid inducers, but we knew that we could not do that in the first generation because the editing may not be ‘complete’ by that point in time—and that we needed to test and retest haploid induction in diverse genotypes in the E1 and E2 generations.

There were many guide RNAs that we could choose from that had appropriate PAM sites, but only a select couple of pairs that could create our desired haploid inducer alleles. Regarding the desired alleles, one way that a pair of guide RNAs could generate such alleles would be for the editing of the first gRNA target site to generate a frameshift which is then restored by a compensatory mutation at the second target site that puts the transcript back into the correct (native) frame. This would result in an altered N-terminal domain amino acid sequence for the intervening string of amino acids between the two guide RNA target sites, and would restore a “native” sequence for the C-terminal domain. Such alleles we decided to call restored frame-shift (“RFS”) alleles. Alternatively, simultaneous or near-simultaneous cutting at both guide RNA target sites could result in a deletion of the intervening nucleic acid sequence. In many cases that deletion would produce a frameshift in the downstream sequence, but in some cases such a deletion could happen to leave the 3′ sequence of the transcript in the normal frame, such that a significant part of the N-terminal domain amino acid sequence is absent from the resulting protein product, but again the C-terminal domain is left intact. We decided to call these large deletion (“LD”) alleles. Finally, in some cases, we designed the gRNAs to target the splice site junctions, and edits at these target sites may generate alternative splicing patterns (for instance, it could lead to intron retention or exon skipping). These alternatively spliced (“AS”) alleles in most cases would lead to premature stop codons and genetic knock outs, but we also found one guide RNA that, if the right edits and the right splicing happened, would not lead to any premature stop codons in frame. Instead it could result in a large insertion in the mature transcript, resulting in a significant alteration in the N-terminal domain by inserting a long stretch of amino acids, but then a specific edit at the second target site could put the sequence back into frame for the C-terminal domain. In other words, with smart design of the gRNAs, splice site mutation may be predicted to generate mature mRNA transcript variants that alter the amino acid code of the N-terminal domain but restore the normal frame and sequence of the C-terminal domain.

In the following examples, we describe in detail the specific guide RNAs and edits we recovered, and the combinations of edits in specific plants. We show in detail how we generated haploid inducer lines in wheat by direct-editing the native CENH3 genes using two guide RNAs targeted to the hypervariable N-terminal domain. As designed, the plants having mutations at both target sites in some cases produced protein products that contained significant alterations to the N-terminal domain amino acid sequence, without affecting the C-terminal domain amino acid sequence. We selected and maintained edited lines that had these type of edited N-terminal tail altered alleles CENH3 alleles and made sure that the other copies of the CenH3 genes (from the A, B, and D genome) were knocked out by mutations produced by those same guide RNAs. We recovered and tested haploid induction in lines that had the desired mutations, including the combination of A genome RFS alleles with B and D genome knockouts. These same lines, with the right combination of edits that we had predicted to generate haploids, indeed led to haploid induction.

### Example 2: Determining the gRNA Sequences to Edit the Fielder Genome's CENH3α Genes

There are two CenH3 genes in hexaploid wheat, TaCen3α and TaCenH3β. The A, B, and D genomes's copies of both genes were cloned in the wheat variety “Fielder” with primers designed against genome sequence of the variety “Chinese Spring_v2”. The sequences are given (SEQ ID NOs: 1-12). Previous studies have shown that viral-induced gene silencing (“VIGS”) of TaCenH3α led to dwarfism and reduced root prolificacy, whereas silencing of TaCenH3β reduced seed set (Yuan et al., New Phytol. 206(2):839-51. 2015). As is the case for most wheat genes, the specific expression patterns and functions of each of the A, B, and D genomes's homologues are not well studied. For genome editing, we opted to modify TaCenH3α in the Fielder spring wheat variety, reasoning that mutations in this gene should not have as much of an impact on seed setting as mutations in TaCenH3β. Homologue-specific Taqman qPCR assays were used to query the expression level of TaCENH3α-A, -B, and -D (SEQ ID NOs: 13-21), in reproductive tissues (pollen, ovary, and anther) as well as juvenile leaf tissue. TaCenH3α-A and -B were expressed at high levels in anthers, pollen and ovaries while the TaCenH3α-D expression transcript was nearly absent (Table 2). In leaf, TaCenH3α-A was the predominant transcript, which may indicate that loss of function of this gene contributes to the dwarf phenotype after TaCenH3α silencing.

TABLE 2
Relative expression of TaCenH3α-A,
TaCenH3α-B, and TaCenH3α-D.

| | Relative expression (Mean ± SD) | | |
|---|---|---|---|
| Tissue | TaCenH3α-A | TaCenH3α-B | TaCenH3α-D |
| Leaf | 1.88 ± 0.36 | 0.00 ± 0.00 | 0.01 ± 0.00 |
| Anther | 44.08 ± 19.62 | 46.66 ± 18.83 | 0.47 ± 0.12 |
| Pollen | 43.62 ± 23.69 | 54.21 ± 17.28 | 4.03 ± 1.38 |
| Ovary | 22.39 ± 11.50 | 15.54 ± 2.73 | 0.19 ± 0.05 |

The guide RNAs were picked using NGG PAM sites and by our predictions of the types of amino acid sequences that would result in the CENH3 protein product if certain edits were made at those target sites. Importantly, most of the guide RNAs that we considered would not able to generate combinations of edits that produced RFS or AS alleles. We focused on selecting the few pairs of gRNAs that could conceivable do so. Guide RNA1 (ACGTCGGCGACACCGGTGCG; SEQ ID NO: 25) (underlined is the approximate site of double stranded break cut induced by the CRISPR-Cas9 complex) is located at the exon 2-intron 2 junction region. This gRNA1 was driven by the TaU6 promoter. Guide RNA2 (CTTGTGGGAGCAGGGGCAAC; SEQ ID NO: 26) targets just after the intron 3-exon 4 junction, driven by TaU6. Guide RNA2 will not edit the 3′ splice acceptor site of intron 3 in most cases. The choice to use two guide RNAs was made so that we could produce significant alterations, e.g., RFS, LDs, or AS alleles, in the N-terminal domain while still leaving the C-terminal domain in frame. For instance, in some plants and edited alleles, both gRNAs will cut at the same time, resulting in a deletion of the intervening sequence. In some cases, the resulting repair will produce a frameshift which will knockout the protein. In other cases, it will produce a shortened LD transcript that lacks intron 2, all of exon 3, and a portion of exon 4, removing approximately the amino acid sequence RAGRAAAPGGAQGA (SEQ ID NO: 76) from the protein product, constituting a significant alteration of the N-terminal domain.

Alternatively, a non-simultaneous cut at both sites could generate a frameshift at gRNA1 (for instance, any indel that hits the coding sequence and is not a multiple of 3) which is restored at the gRNA2 site by a complimentary indel, thus putting the coding sequence back in its normal frame. For example, a 1 nucleotide (“nt”) deletion at gRNA1 and a 1 nt insertion at gRNA2 would restore the coding frame, leading to an RFS allele. This allele would likely not be a loss of function, assuming there are no stop codons generated in the intervening frameshifted sequence. From our evaluation of the potential changes, we could see that at least one combination of edits would generate an RFS between gRNA1 and gRNA2 that did not have any stop codon in the intervening sequence. Thus, we could predict which pairs of small indels at gRNA1 and gRNA2 would combine to give us a functional RFS allele.

The two guide RNAs could generate alternatively spliced (“AS”) alleles that also have the capacity to act as RFS or large insertion alleles. Guide RNA1 will cut between the GT (SEQ ID NO: 25; underlined above). That is the 5′ splice donor site at the end of exon 2. AS alleles could be generated if the GT is modified such that intron 2 is not correctly spliced, leading to the retention of intron 2 in the coding sequence. Upon translation, the ribosome would read through this intron and generate a novel insertion of 44-47 amino acids, depending on the nature of the indels at gRNA1 and gRNA2. This novel insertion can be predicted by reading the new coding frame after factoring in the indels and the translation of the new mature mRNA. For instance, if gRNA1 and gRNA2 generate insertions of a single A nucleotide at both sites of TaCENH3α-A, the transcript may be alternatively spliced leading to an insertion of the amino acid sequence “VARDLPGSLPFRFVLFSVFWSDLLVTCSTECRGEPGGRRPQGGLKGQ” (SEQ ID NO: 77) with removal of the WT sequence “RRAGRAAAPGGAQGA” (SEQ ID NO: 76) from exon 3 before the normal sequence is restored by the gRNA mutation. Likewise, a different mutation at gRNA1 (for example, the deletion of GTG) combined with gRNA2 (deletion of a C) can similarly be predicted to cause alternative splicing to generate a novel insertion of “GTFPGRFLFVSSCFLFFGLTCSSPVRRNAEASRAGGGPRGGSRG” (SEQ ID NO: 78) with the removal of the native sequence “RAGRAAAPGGAQGA” (SEQ ID NO: 76). We can also predict that other mutation combinations at gRNA1 and gRNA2 would generate frameshifts that are not put back into frame, leading to loss of function alleles. Similarly, alternative spliced alleles induced at other sites in the N-terminal region of CenH3 would not be able to generate modified mRNA sequences with a C-terminal domain restored to the normal amino acid sequence, because there would be stop codons generated in the introns retained, or after skipping one or more exons. Therefore, the gRNAs can be selected specifically for their capacity to generate large changes in the N-terminal domain while leaving the C-terminal domain intact and translated normally.

### Example 3: Construct Design and Plant Transformation

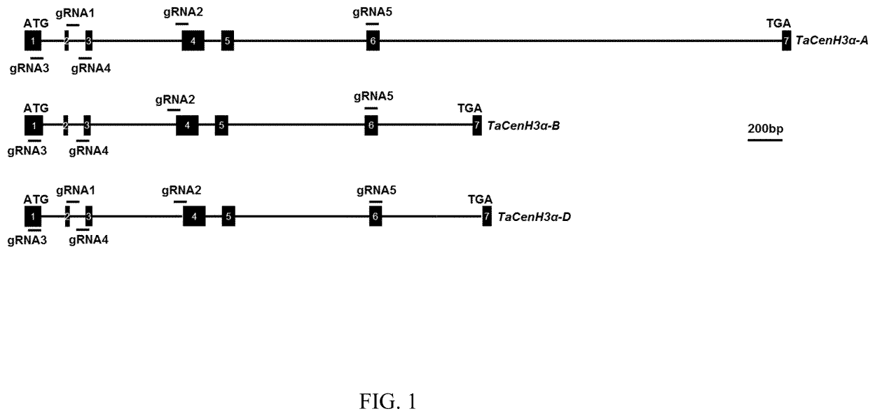

After cloning the specific sequences of TaCENH3α-A, -B, and -D in the Fielder variety with primers designed against genome sequence of Chinese Spring, we selected the gRNAs according to PAM sites. gRNA1 (ACGTCGGCGACACCGGTGCG; SEQ ID NO: 25) locates in exon 2-intron 2 junction region (FIG. 1). gRNA2 (CTTGTGGGAGCAGGGGCAAC; SEQ ID NO: 26) targets just after the intron 3-exon 4 junction. SpCas9 gene was wheat codon-optimized with two NLSs at both ends and driven by sugarcane Ubi promoter with two enhancers. The gRNA cassettes including the wheat U6 promoter and gRNA scaffolds was synthesized by GenScript (www.genscript.com) and cloned into a binary vector, Construct 24194 (SEQ ID NO: 74).

Fielder was used for transformation, a spring wheat inbred. Immature embryos about 2.0-2.5 mm in diameter were harvested, sterilized with 70% ethanol for 1 min and 1% sodium hypochlorite for 10 min. After sterilization, immature embryos were isolated by scalpel and spatula into a small tube and centrifuged at 20,000×g at 4° C. for 10 min in inoculation medium. The isolated embryos were infected with Agrobacterium for 5 min, then transferred to co-cultivation medium at 23° C. in the dark for 2 days. The embryo axis was excised from the immature embryos before transferring to resting medium, cultured at 25° C. in the dark for 5 days, then transferred to selection medium containing mannose 15 g/L. See Y. Ishida, et al., Wheat (Triticum aestivum L.) Transformation Using Immature Embryos, METHODS IN MOLECULAR BIOLOGY 1223: 189-198 (2015). After 4 weeks, the vigorously grown calli were transferred to regeneration media to generate green plants. Surviving plants went through Taqman check, which analyzed the presence or absence of DNA segments from the transgenic DNA insertion; of these, only plants positive for 35S and PMI Taqman assays were sent to the greenhouse.

### Example 4: Sequencing E0 Edited Plants

Genomic DNA was isolated from juvenile leaves of Tagman positive E0 plants. Sequencing was performed with high fidelity DNA polymerase, namely KOD-Plus-Neo (source: TOYOBO Life Science). TaCenH3α-A allele specific primers were used (FA, SEQ ID NO: 50; R3, SEQ ID NO: 51). PCR was performed as follows: 95° C. 5 min; 35 cycles of 95° C. 30 sec, 65° C. 30 sec, 68° C. 1 min; 68° C. 10 min. PCR reaction mixture comprises 11.5 μdistilled water, 2.5 μl 10×PCR buffer for KOD-Plus-Neo, 1 μl 2 mM dNTPs, 1 μl 25 mM MgSO₄, 1 μl KOD-Plus-Neo DNA polymerase, 1 μl forward primer FA (10 μM), 1 μl reverse primer R3 (10 μM), and 1 μl genomic DNA. PCR products were sequenced directly via SQ-1 primer (SEQ ID NO: 79) or cloned into pEASY-Blunt Zero cloning vector (Transgen Biotech). M13R (SEQ ID NO: 52) and M13F (SEQ ID NO: 53) were used for colony sequencing.

### Example 5: Wheat Event A004A

The wheat event A004A exhibited haploid induction. The event number A004A is one of hundreds of transgenic plants that were produced via transformation of construct 24194. Tagman assay followed by direct sequencing indicated that the genotype for the TaCENH3α genes were AA*BBdd at E0 seedling stage. Here, a capital letter indicates a wild-type TaCENH3α allele without editing, a lower-case letter indicates a loss-of-function of allele, and a capital letter with an asterisk (*) indicates a restored frame shift (RFS), large deletion (LD) or alternatively spliced (AS) allele, which means a putative haploid inducer allele. The A004A plant A* allele contains an adenine insertion at the target site for gRNA 1, and another adenine insertion at the target site of gRNA2 (SEQ ID NO:56). The adenine insertion at gRNA1 is actually in the intron, 3 bp downstream from the end of Exon 2, and right after the 5′ splice junction. It does not itself disrupt the coding sequence, but it may alter the splicing pattern in some instances. The adenine insertion at gRNA2 is in Exon 3, and shifts the frame of the coding sequence. Prediction of the splicing pattern induced by the insertion of an Adenine at the gRNA1 target site indicates that this may be an AS allele that exhibits intron retention (IR) of intron number 2, because having an extra adenine after the 5′ splice donor site can alter the initiation of intron removal, triggering alternative splicing. Alternative splicing in this case would to an insertion of many amino acids leading into exon 3. If there were alternative splicing, analysis of the outcome indicates that the sequences in exon 3 would be out of frame until the gRNA2 edit, which is another insertion of an adenine, restores the normal frame and amino acid code for the C-terminal domain.

To verify that the A004A A* allele is alternatively spliced and produces a putative haploid inducer allele, we examined the mature mRNA sequences of the CENH3α-A gene in A004A juvenile stage E0 leaf. Total RNA of juvenile leaves was extracted using INVITROGEN TRIzol following manufacturer's instructions. cDNA was synthesized from 1 mg of total RNA via Superscript III first-strand synthesis system (Invitrogen) with oligo-dT primer. KOD-Plus-Neo (TOYOBO) was used to amplify TaCenH3α-A transcripts with primers F1 (SEQ ID NO:54) and R1 (SEQ ID NO:55). PCR performed according to manufacturers instructions and as follows: 95° C. 5 min; 35 cycles of 95° C. 30 sec, 62° C. 20 sec, 68° C. 20 sec, 35 cycles; 68° C. 10 min. PCR reaction mixture comprises 11.5 μl distilled water, 2.5 μl 10×PCR buffer for KOD-Plus-Neo, 1 μl 2 mM dNTPs, 1 μl 25 mM MgSO₄, 1 μl KOD-Plus-Neo, 1 μl F1 primer (10 μM), 1 μl R1 primer (10 μM), and 1 μl cDNA. PCR product was purified by GeneJET PCR Purification Kit (Thermo Scientific) and cloned into pEASY-Blunt Zero cloning vector (Transgen Biotech). Primers M13R and M13F were used for colony sequencing. Several clones per PCR product were sequenced and analyzed by Vector NTI software (Invitrogen). Relative expression of splicing variants were calculated by number of clones. Analysis of the PCR sequencing of the colonies indicated the TaCENH3α-A mRNA in A004A has two transcripts, indicative of alternative splicing. One of the transcripts (SEQ ID NO:58), found in 8 out of 18 (44%) of colonies, was spliced using the canonical 5′ splice site. For these transcripts, normal splicing of Intron 2 means that the gRNA1 edit did not impact the amino acid translation of the mature mRNA; however, the gRNA2 edit caused a frame-shift. So, in this instance the constitutively spliced mature mRNAs are actually knockout transcripts. On the other hand, 10 out of 18 (56%) of the colonies had mature mRNA transcripts with intron 2 retained (SEQ ID NO:57), leading to an N terminal RFS allele which contains 47 new amino acids, thus 32 amino acids were inserted from intron 2 along with 15 amino acids that are altered by the frameshift in exon 3. This 32aa insertion and 47aa overall change severely alters the N terminal domain of the proteins produced by translation of the RFS mRNA. Importantly, the mature mRNA sequence that we obtained in our AS allele A004A—TaCENH3α-A* is the exact mature mRNA sequence outcome that we predicted would happen. A004A E0 plant was maintained through flowering. We did not observe any abnormal phenotypes, and the E1 seeds (after self-pollination) were harvested from the spikes produced by this plant.

The ploidy level of E1 progeny seeds, produced by self-pollination of the A004A plant, was checked. The seeds were planted and the seedlings that germinated were sampled and analyzed for DNA content by flow cytometry. Haploids were obtained in the first batch of progeny plants sowed: the haploid induction rate (“HIR”) was 3.8%. In the second batch of seedlings, haploids were observed again, and the HIR was 4.2%. Wheat haploids are smaller than their diploid counterparts, similar to haploids in rice and corn. Importantly, these plants either did not have both copies of the “B” allele knocked out, or did not have a restored frameshift induced by a mutation at gRNA2 target site. This suggests that only the right combination of edits at both the target sites at gRNA1 and gRNA2 in the CENH3α-A gene, when paired with a knockout of both copies of the “B” allele, is sufficient to trigger haploid induction.

In the first batch, we observed two twin-seedling plants; both seedlings were haploids based on flow cytometry check. This indicates there are two haploid embryos in one seed. Twin embryos may be caused by a disruption of ovule development, which may be triggered in part by the edits in CENH3α, although more experimental work is needed to confirm this.

While we were slightly surprised that we observed haploids after self-pollination (because normally haploids are only induced in CENH3 modifications during outcross), the continuous capacity for editing the E0 plants mean that the male and female sex cells may inherit different sequences (edits) and thus have different centromere binding and kinetochore construction than each other, leading to haploidy after selfing.

### Example 6: Wheat Event C003A

Plant C003A is edited such that the TaCenH3α genotype is A*abbdd at E0 plant stage. A* is introduced by deleting a guanine in gRNA1 and inserting an adenine in gRNA2. At the protein level, there is an eleven amino acid difference in N-terminal domain compared to the wildtype sequence. SEQ ID NOs: 63-65 show the A* genomic CenH3 sequence, the A* CDS sequence, and the A* protein sequence, respectively, for C003A.

E1 seeds were produced by selfing C003A E0 plant. E1 plants with A*A*bbdd genotype were grown in the greenhouse to determine its ability to induce haploids upon outcross. A wildtype plant (Tester 03S0352-22) was selected as pollen donor. E1 C003A was manually emasculated, and pollinated with the wildtype pollen. Haploids were detected by SNP markers (SEQ ID #29-43), which can tell difference between Fielder and 03S0352-22, then confirmed by flow cytometry check. In 208 F1 plants, we obtained one haploid. This showed paternal-only genotypes for four markers but maternal genotype for one marker (KW 11091).

### Example 7: Wheat Event A073A

Plant A073A had the genotype AA*B*bdd for TaCENH3α at the E0 seedling stage. The A073A A* allele has an adenine insertion, caused by gRNA 3, and a guanine deletion, caused by gRNA4 in the genomic DNA. This triggers a restored frameshift at the protein level, with a thirty-one amino acid difference between the wild type and edited versions in the N-terminal domain. This plant was highlighted as a potential progenitor of haploid inducer lines because of its capacity to generate offspring that were A*A*bbdd—an ideal genetic combination for triggering haploid induction. SEQ ID NOs: 69-71 show the A* genomic CenH3 sequence, the A* CDS sequence, and the A* protein sequence, respectively, for A073A.

E1 seeds were produced by allowing self-pollination of the A073A E0 plant. E1 plants were sequenced and those with the genotypic combination AA*bbdd were selected to be grown further in the greenhouse for determining haploid induction potential upon outcrossing. Using the tester line 03S0352-22 selected as the pollen donor, E1 edited plants were manually emasculated and hand pollinated. Haploids were detected by SNP markers that can distinguish Fielder and 03S0352-22 genotypically. The putative haploids, as identified by homozygousity for these markers, were then confirmed by flow cytometry check of total DNA content. Among 57 F1 plants, 53 were predominantly heterozygous for the SNP markers and were diploid by ploidy check, indicating that they were hybrids. In contrast, four had only paternal genomic SNP markers and were haploids by flow cytometry, amounting to a 7% outcross haploid induction rate.

E1 plants with AA*bbdd also led to E2 haploids during selfing (Table 3). We observed one haploid from 13 E2 plants (a 7.7% haploid induction rate). Meanwhile we observed several plants with partially chromosome elimination.

TABLE 3
Ploidy level of selfed E2 plants derived from A073A.

| F1 plant ID | Ploidy |
|---|---|
| 001-11 | 1n + X (Aneuploidy) |
| 001-13 | 1n + X (Aneuploidy) |
| 001-14 | 1n + X (Aneuploidy) |
| 001-17 | 1n + X (Aneuploidy) |
| 001-18 | 1n + X (Aneuploidy) |
| 001-19 | 2n (Diploid) |
| 001-22 | 1n + X (Aneuploidy) |
| 001-23 | 1n + X (Aneuploidy) |
| 001-24 | 2n (Diploid) |
| 001-26 | 1n + X (Aneuploidy) |
| 001-27 | 2n (Diploid) |
| 001-28 | 1n (Haploid) |
| 001-30 | 2n (Diploid) |

1-10. (canceled)

11. A method of generating a haploid-inducing wheat plant, the method comprising: a. obtaining at least a wheat plant cell comprising an A genome, a B genome, and a D genome; b. mutating the B genome to obtain a homozygous knock-out mutation in a CENH3α gene; c. mutating the D genome to obtain a homozygous knock-down mutation in a CENH3α gene; and d. generating a wheat plant therefrom; whereby the wheat plant generated from step (d) produces haploid progeny when crossed with a wildtype wheat plant.

12. (canceled)

13. (canceled)

14. The method of claim 11, wherein the A genome comprises a knock-down mutation in a CENH3α gene.

15. The method of claim 14, wherein the knock-down mutation in the CENH3α gene is a restored frame shift mutation.

16. The method of claim 15, wherein the restored frame shift mutation is selected from the group consisting of SEQ ID NO: 56, a nucleic acid sequence 70% identical to SEQ ID NO: 56, SEQ ID NO: 63, a nucleic acid sequence 70% identical to SEQ ID NO: 63, SEQ ID NO: 69, and a nucleic acid sequence 70% identical to SEQ ID NO: 69, or a combination thereof.

17. A wheat plant comprising a mutated CENH3α gene comprising at least one deletion mutation in the N-terminal domain resulting in a frame shift, a restored frame shift, or a large deletion.

18. A wheat plant comprising a mutated CENH3α gene comprising at least one insertion mutation in the N-terminal domain resulting in a frame shift, a restored frame shift, or a large deletion.

19-29. (canceled)


SEQUENCE LISTING (167 sequences)

SEQ ID NO: 1 - skipped

SEQ ID NO: 2 - RNA, 20 nt, synthetic construct
  source 1..20: mol_type = other RNA
  misc_feature 1..20: note = primer or gRNA
acgtcggcgacaccggtgcg

SEQ ID NO: 3 - RNA, 20 nt, synthetic construct
  source 1..20: mol_type = other RNA
  misc_feature 1..20: note = primer or gRNA
cttgtgggagcaggggcaac

SEQ ID NO: 4 - RNA, 20 nt, synthetic construct
  source 1..20: mol_type = other RNA
  misc_feature 1..20: note = primer or gRNA
acgtcggcgacaccggtgcg

SEQ ID NO: 5 - RNA, 20 nt, synthetic construct
  source 1..20: mol_type = other RNA
  misc_feature 1..20: note = primer or gRNA
cttgtgggagcaggggcaac

SEQ ID NO: 6 - RNA, 20 nt, synthetic construct
  misc_feature 1..20: note = primer or gRNA
  source 1..20: mol_type = other RNA
acgtcggcgacaccggtgcg

SEQ ID NO: 7 - RNA, 20 nt, synthetic construct
  misc_feature 1..20: note = primer or gRNA
  source 1..20: mol_type = other RNA
cttgtgggagcaggggcaac

SEQ ID NO: 8 - DNA, 15 bp, synthetic construct
  source 1..15: mol_type = other DNA
  misc_feature 1..15: note = primer or gRNA
tggcccgcaccaagc

SEQ ID NO: 9 - DNA, 23 bp, synthetic construct
  misc_feature 1..23: note = primer or gRNA
  source 1..23: mol_type = other DNA
gctgtatgtccttttgcatgacg

SEQ ID NO: 10 - DNA, 20 bp, synthetic construct
  source 1..20: mol_type = other DNA
  misc_feature 1..20: note = primer or gRNA
agcgaatgaaacaacacacg

SEQ ID NO: 11 - DNA, 20 bp, synthetic construct
  misc_feature 1..20: note = primer or gRNA
  source 1..20: mol_type = other DNA
gaagtcggtgacctccttga

SEQ ID NO: 12 - DNA, 20 bp, synthetic construct
  source 1..20: mol_type = other DNA
  misc_feature 1..20: note = primer or gRNA
caaggaggtcaccgacttct

SEQ ID NO: 13 - DNA, 20 bp, synthetic construct
  source 1..20: mol_type = other DNA
  misc_feature 1..20: note = primer or gRNA
gccttgcaagctgtatgtcc

SEQ ID NO: 14 - DNA, 20 bp, synthetic construct
  source 1..20: mol_type = other DNA
  misc_feature 1..20: note = primer or gRNA
actagacgagtcgggacgaa

SEQ ID NO: 15 - DNA, 20 bp, synthetic construct
  misc_feature 1..20: note = primer or gRNA
  source 1..20: mol_type = other DNA
gaagtcggtgacctccttga

SEQ ID NO: 16 - DNA, 20 bp, synthetic construct
  misc_feature 1..20: note = primer or gRNA
  source 1..20: mol_type = other DNA
caaggaggtcaccgacttct

SEQ ID NO: 17 - DNA, 18 bp, synthetic construct
  misc_feature 1..18: note = primer or gRNA
  source 1..18: mol_type = other DNA
acgcctcgcgagctgtat

SEQ ID NO: 18 - DNA, 20 bp, synthetic construct
  source 1..20: mol_type = other DNA
  misc_feature 1..20: note = primer or gRNA
gggtccaagaaagacacacg

SEQ ID NO: 19 - DNA, 20 bp, synthetic construct
  source 1..20: mol_type = other DNA
  misc_feature 1..20: note = primer or gRNA
gaagtcggtgacctccttga

SEQ ID NO: 20 - DNA, 20 bp, synthetic construct
  source 1..20: mol_type = other DNA
  misc_feature 1..20: note = primer or gRNA
caaggaggtcaccgacttct

SEQ ID NO: 21 - DNA, 18 bp, synthetic construct
  source 1..18: mol_type = other DNA
  misc_feature 1..18: note = primer or gRNA
acgcctcgcgagctgtat

SEQ ID NO: 22 - DNA, 31 bp, synthetic construct
  source 1..31: mol_type = other DNA
  misc_feature 1..31: note = primer or gRNA
caaaacagataaaacaaaatgtagggaatgt

SEQ ID NO: 23 - DNA, 23 bp, synthetic construct
  source 1..23: mol_type = other DNA
  misc_feature 1..23: note = primer or gRNA
ggtgcaaacgggatgagaaagtc

SEQ ID NO: 24 - DNA, 20 bp, synthetic construct
  misc_feature 1..20: note = primer or gRNA
  source 1..20: mol_type = other DNA
cgactcgcctcgcgctagta

SEQ ID NO: 25 - DNA, 17 bp, synthetic construct
  source 1..17: mol_type = other DNA
  misc_feature 1..17: note = primer or gRNA
gtaaaacgacggccagt

SEQ ID NO: 26 - DNA, 17 bp, synthetic construct
  misc_feature 1..17: note = primer or gRNA
  source 1..17: mol_type = other DNA
caggaaacagctatgac

SEQ ID NO: 27 - DNA, 15 bp, synthetic construct
  source 1..15: mol_type = other DNA
  misc_feature 1..15: note = primer or gRNA
tggcccgcaccaagc

SEQ ID NO: 28 - DNA, 23 bp, synthetic construct
  source 1..23: mol_type = other DNA
  misc_feature 1..23: note = primer or gRNA
gctgtatgtccttttgcatgacg

SEQ ID NO: 29 - DNA, 17 bp, synthetic construct
  source 1..17: mol_type = other DNA
  misc_feature 1..17: note = primer or gRNA
ggttcaggccaggcacg

SEQ ID NO: 30 - DNA, 19 bp, synthetic construct
  misc_feature 1..19: note = primer or gRNA
  source 1..19: mol_type = other DNA
caaatggtgcaaacgggat

SEQ ID NO: 31 - DNA, 15 bp, synthetic construct
  misc_feature 1..15: note = primer or gRNA
  source 1..15: mol_type = other DNA
aggtatcagaagtcg

SEQ ID NO: 32 - DNA, 30 bp, synthetic construct
  misc_feature 1..30: note = primer or gRNA
  source 1..30: mol_type = other DNA
gtcgttagaaagtattgtaggtgtatcatt

SEQ ID NO: 33 - DNA, 23 bp, synthetic construct
  misc_feature 1..23: note = primer or gRNA
  source 1..23: mol_type = other DNA
ctgaatgcaaaagtcgaatgatc

SEQ ID NO: 34 - DNA, 19 bp, synthetic construct
  source 1..19: mol_type = other DNA
  misc_feature 1..19: note = primer or gRNA
tagatgtgtcttcaaagtt

SEQ ID NO: 35 - DNA, 21 bp, synthetic construct
  source 1..21: mol_type = other DNA
  misc_feature 1..21: note = primer or gRNA
cgttcgactcgctggagtagt

SEQ ID NO: 36 - DNA, 23 bp, synthetic construct
  source 1..23: mol_type = other DNA
  misc_feature 1..23: note = primer or gRNA
cctgccgattgtttgttttactg

SEQ ID NO: 37 - DNA, 24 bp, synthetic construct
  source 1..24: mol_type = other DNA
  misc_feature 1..24: note = primer or gRNA
tagtagtaaccgcgcctcccgcac

SEQ ID NO: 38 - DNA, 4396 bp, Triticum aestivum
  source 1..4396: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacaccggtgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggggctgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 39 - RNA, 489 nt, Triticum aestivum
  source 1..489: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccgaggcgagccgggcgggcggcggccccagggggggctcaaggggctgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 40 - protein, 162 aa, Triticum aestivum
MARTKHPAVRKTKAPPKKQLGPRPAQRRQETDGAGTSATPRRAGRAAAPGGAQGAGQPKQRKPHRFRPGTVALREIRRYQKSVDFLIPFAPFVRLIKEVTDFFCPEISRWTPQALVAIQEAAEYHLVDVFERANHCAIHAKRVTVMQKDIQLARRIGGRRLW

SEQ ID NO: 41 - DNA, 4396 bp, Triticum aestivum
  source 1..4396: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacacgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggggcaaactgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 42 - RNA, 583 nt, Triticum aestivum
  source 1..583: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacacgcgcgggaccttcccgggtcgcttccttttcgtttcgtcttgttttctgttttttggtctgacttgctcgtcacctgttcgacggaatgcagaggcgagccgggcgggcggcggccccagggggggctcaaggggcaaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 43 - protein, 59 aa, Triticum aestivum
MARTKHPAVRKTKAPPKKQLGPRPAQRRQETDGAGTSATRAGPSRVASFSFRLVFCFLV

SEQ ID NO: 44 - DNA, 4396 bp, Triticum aestivum
  source 1..4396: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacactgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggggcaactgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 45 - RNA, 583 nt, Triticum aestivum
  source 1..583: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacactgcgcgggaccttcccgggtcgcttccttttcgtttcgtcttgttttctgttttttggtctgacttgctcgtcacctgttcgacggaatgcagaggcgagccgggcgggcggcggccccagggggggctcaaggggcaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 46 - protein, 136 aa, Triticum aestivum
MARTKHPAVRKTKAPPKKQLGPRPAQRRQETDGAGTSATLRGTFPGRFLFVSSCFLFFGLTCSSPVRRNAEASRAGGGPRGGSRGNWATQAEETTPVQARHGGTAGDQEVSEVGRLSHPVCTICPSDQGGHRLLLS

SEQ ID NO: 47 - DNA, 4396 bp, Triticum aestivum; identical to SEQ ID NO: 38
  source 1..4396: mol_type = other DNA

SEQ ID NO: 48 - RNA, 489 nt, Triticum aestivum; identical to SEQ ID NO: 39
  source 1..489: mol_type = other RNA

SEQ ID NO: 49 - protein, 162 aa, Triticum aestivum; identical to SEQ ID NO: 40

SEQ ID NO: 50 - DNA, 4384 bp, Triticum aestivum
  source 1..4384: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacaccggcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgaactgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 51 - RNA, 562 nt, Triticum aestivum
  source 1..562: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccggcgcgggaccttcccgggtcgcttccttttcgtttcgtcttgttttctgtttttttggtctgacttgctcgtcacctgttcgacggaatgcagaggcgagccgggcgggcggcggccccagggggggctcaagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 52 - protein, 60 aa, Triticum aestivum
MARTKHPAVRKTKAPPKKQLGPRPAQRRQETDGAGTSATPARDLPGSLPFRFVLFSVFLV

SEQ ID NO: 53 - DNA, 4401 bp, Triticum aestivum
  source 1..4401: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacaccggttgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggggctaactgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 54 - RNA, 587 nt, Triticum aestivum
  source 1..587: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccggttgcgcgggaccttcccgggtcgcttccttttcgtttcgtcttgttttctgttttttggtctgacttgctcgtcacctgttcgacggaatgcagaggcgagccgggcgggcggcggccccagggggggctcaaggggctaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtattgaaagggcaaatcactgtgccatccatgcaaagcgtgttaccgtcatgcaaaaggacatacagcttgcaaggcgtatcggcgggaggaggctttggtga

SEQ ID NO: 55 - protein, 195 aa, Triticum aestivum
MARTKHPAVRKTKAPPKKQLGPRPAQRRQETDGAGTSATPVARDLPGSLPFRFVLFSVFWSDLLVTCSTECRGEPGGRRPQGGLKGLTGQPKQRKPHRFRPGTVALREIRRYQKSVDFLIPFAPFVRLIKEVTDFFCPEISRWTPQALVAIQEAAEYHLVDVLKGQITVPSMQSVLPSCKRTYSLQGVSAGGGFG

SEQ ID NO: 56 - RNA, 512 nt, Triticum aestivum
  source 1..512: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccggttgcgcgggaccttcccggaggcgagccgggcgggcggcggccccagggggggctcaaggggctaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtattgaaagggcaaatcactgtgccatccatgcaaagcgtgttaccgtcatgcaaaaggacatacagcttgcaaggcgtatcggcgggaggaggctttggtga

SEQ ID NO: 57 - protein, 170 aa, Triticum aestivum
MARTKHPAVRKTKAPPKKQLGPRPAQRRQETDGAGTSATPVARDLPGGEPGGRRPQGGLKGLTGQPKQRKPHRFRPGTVALREIRRYQKSVDFLIPFAPFVRLIKEVTDFFCPEISRWTPQALVAIQEAAEYHLVDVLKGQITVPSMQSVLPSCKRTYSLQGVSAGGGFG

SEQ ID NO: 58 - DNA, 4401 bp, Triticum aestivum
  source 1..4401: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacaccggttgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggggcaaactgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 59 - RNA, 588 nt, Triticum aestivum
  source 1..588: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccggttgcgcgggaccttcccgggtcgcttccttttcgtttcgtcttgttttctgttttttggtctgacttgctcgtcacctgttcgacggaatgcagaggcgagccgggcgggcggcggccccagggggggctcaaggggcaaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 60 - protein, 195 aa, Triticum aestivum
MARTKHPAVRKTKAPPKKQLGPRPAQRRQETDGAGTSATPVARDLPGSLPFRFVLFSVFWSDLLVTCSTECRGEPGGRRPQGGLKGQTGQPKQRKPHRFRPGTVALREIRRYQKSVDFLIPFAPFVRLIKEVTDFFCPEISRWTPQALVAIQEAAEYHLVDVFERANHCAIHAKRVTVMQKDIQLARRIGGRRLW

SEQ ID NO: 61 - RNA, 513 nt, Triticum aestivum
  source 1..513: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccggttgcgcgggaccttcccggaggcgagccgggcgggcggcggccccagggggggctcaaggggcaaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 62 - protein, 170 aa, Triticum aestivum
MARTKHPAVRKTKAPPKKQLGPRPAQRRQETDGAGTSATPVARDLPGGEPGGRRPQGGLKGQTGQPKQRKPHRFRPGTVALREIRRYQKSVDFLIPFAPFVRLIKEVTDFFCPEISRWTPQALVAIQEAAEYHLVDVFERANHCAIHAKRVTVMQKDIQLARRIGGRRLW

SEQ ID NO: 63 - DNA, 4249 bp, Triticum aestivum
  source 1..4249: mol_type = other DNA
atggcccgcaccaagcacccggccgtcaggaagaccaaggcgccgcccaagaagcagctcgggccccgccccgcgcagcggcggcaggagacaggttcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacaccggtgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcaactgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 64 - RNA, 591 nt, Triticum aestivum
  source 1..591: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccggttgcgcgggaccttcccgggtcgcttccttttcgtttcgtcttgttttctgttttttggtctgacttgctcgtcacctgttcgacggaatgcagaggcgagccgggcgggcggcggccccagggggggctcaagttttcttcaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 65 - protein, 196 aa, Triticum aestivum
MARTKHPAVRKTKAPPKKQLGPRPAQRRQETDGAGTSATPVARDLPGSLPFRFVLFSVFWSDLLVTCSTECRGEPGGRRPQGGLKFSSTGQPKQRKPHRFRPGTVALREIRRYQKSVDFLIPFAPFVRLIKEVTDFFCPEISRWTPQALVAIQEAAEYHLVDVFERANHCAIHAKRVTVMQKDIQLARRIGGRRLW

SEQ ID NO: 66 - RNA, 609 nt, Triticum aestivum
  source 1..609: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccggttgcgcgggaccttcccgggtcgcttccttttcgtttcgtcttgttttctgttttttggtctgacttgctcgtcacctgttcgacggaatgcagaggcgagccgggcgggcggcggccccagggggggctcaagagggtagtggatcggcagttttcttcaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 67 - protein, 202 aa, Triticum aestivum
MARTKHPAVRKTKAPPKKQLGPRPAQRRQETDGAGTSATPVARDLPGSLPFRFVLFSVFWSDLLVTCSTECRGEPGGRRPQGGLKRVVDRQFSSTGQPKQRKPHRFRPGTVALREIRRYQKSVDFLIPFAPFVRLIKEVTDFFCPEISRWTPQALVAIQEAAEYHLVDVFERANHCAIHAKRVTVMQKDIQLARRIGGRRLW

SEQ ID NO: 68 - RNA, 534 nt, Triticum aestivum
  source 1..534: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccggttgcgcgggaccttcccggaggcgagccgggcgggcggcggccccagggggggctcaagagggtagtggatcggcagttttcttcaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 69 - protein, 177 aa, Triticum aestivum
MARTKHPAVRKTKAPPKKQLGPRPAQRRQETDGAGTSATPVARDLPGGEPGGRRPQGGLKRVVDRQFSSTGQPKQRKPHRFRPGTVALREIRRYQKSVDFLIPFAPFVRLIKEVTDFFCPEISRWTPQALVAIQEAAEYHLVDVFERANHCAIHAKRVTVMQKDIQLARRIGGRRLW

SEQ ID NO: 70 - DNA, 4401 bp, Triticum aestivum; identical to SEQ ID NO: 58
  source 1..4401: mol_type = other DNA

SEQ ID NO: 71 - RNA, 588 nt, Triticum aestivum; identical to SEQ ID NO: 59
  source 1..588: mol_type = other RNA

SEQ ID NO: 72 - protein, 195 aa, Triticum aestivum; identical to SEQ ID NO: 60

SEQ ID NO: 73 - RNA, 513 nt, Triticum aestivum; identical to SEQ ID NO: 61
  source 1..513: mol_type = other RNA

SEQ ID NO: 74 - protein, 170 aa, Triticum aestivum; identical to SEQ ID NO: 62

SEQ ID NO: 75 - DNA, 4397 bp, Triticum aestivum
  source 1..4397: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacactgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggggcaaactgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 76 - RNA, 584 nt, Triticum aestivum
  source 1..584: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacactgcgcgggaccttcccgggtcgcttccttttcgtttcgtcttgttttctgttttttggtctgacttgctcgtcacctgttcgacggaatgcagaggcgagccgggcgggcggcggccccagggggggctcaaggggcaaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 77 - protein, 126 aa, Triticum aestivum
MARTKHPAVRKTKAPPKKQLGPRPAQRRQETDGAGTSATLRGTFPGRFLFVSSCFLFFGLTCSSPVRRNAEASRAGGGPRGGSRGKLGNPSRGNHTGSGQARWHCGRSGGIRSRSTFSSRLHHLSV

SEQ ID NO: 78 - DNA, 4396 bp, Triticum aestivum; identical to SEQ ID NO: 44
  source 1..4396: mol_type = other DNA

SEQ ID NO: 79 - RNA, 583 nt, Triticum aestivum; identical to SEQ ID NO: 45
  source 1..583: mol_type = other RNA

SEQ ID NO: 80 - protein, 136 aa, Triticum aestivum; identical to SEQ ID NO: 46

SEQ ID NO: 81 - DNA, 4401 bp, Triticum aestivum
  source 1..4401: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacaccggtagcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggggcaaactgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 82 - RNA, 588 nt, Triticum aestivum
  source 1..588: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccggtagcgcgggaccttcccgggtcgcttccttttcgtttcgtcttgttttctgttttttggtctgacttgctcgtcacctgttcgacggaatgcagaggcgagccgggcgggcggcggccccagggggggctcaaggggcaaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 83 - protein, 195 aa, Triticum aestivum; identical to SEQ ID NO: 60

SEQ ID NO: 84 - DNA, 4400 bp, Triticum aestivum
  source 1..4400: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacaccggtgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggggcaaactgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 85 - RNA, 493 nt, Triticum aestivum
  source 1..493: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccgaggcgagccgggcgggcggcggccccagggggggctcaaggggcaaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 86 - protein, 106 aa, Triticum aestivum
MARTKHPAVRKTKAPPKKQLGPRPAQRRQETDGAGTSATPRRAGRAAAPGGAQGANWATQAEETTPVQARHGGTAGDQEVSEVGRLSHPVCTICPSDQGGHRLLLS

SEQ ID NO: 87 - DNA, 4399 bp, Triticum aestivum
  source 1..4399: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacaccggtgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggggcaactgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 88 - RNA, 492 nt, Triticum aestivum
  source 1..492: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccgaggcgagccgggcgggcggcggccccagggggggctcaaggggcaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 89 - protein, 163 aa, Triticum aestivum
MARTKHPAVRKTKAPPKKQLGPRPAQRRQETDGAGTSATPRRAGRAAAPGGAQGATGQPKQRKPHRFRPGTVALREIRRYQKSVDFLIPFAPFVRLIKEVTDFFCPEISRWTPQALVAIQEAAEYHLVDVFERANHCAIHAKRVTVMQKDIQLARRIGGRRLW

SEQ ID NO: 90 - DNA, 4400 bp, Triticum aestivum
  source 1..4400: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacaccggttgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggggcaactgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 91 - RNA, 587 nt, Triticum aestivum
  source 1..587: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccggttgcgcgggaccttcccgggtcgcttccttttcgtttcgtcttgttttctgttttttggtctgacttgctcgtcacctgttcgacggaatgcagaggcgagccgggcgggcggcggccccagggggggctcaaggggcaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 92 - protein, 127 aa, Triticum aestivum
MARTKHPAVRKTKAPPKKQLGPRPAQRRQETDGAGTSATPVARDLPGSLPFRFVLFSVFWSDLLVTCSTECRGEPGGRRPQGGLKGQLGNPSRGNHTGSGQARWHCGRSGGIRSRSTFSSRLHHLSV

SEQ ID NO: 93 - DNA, 4391 bp, Triticum aestivum
  source 1..4391: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacaccggtgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 94 - RNA, 484 nt, Triticum aestivum
  source 1..484: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccgaggcgagccgggcgggcggcggccccagggggggctcaaggggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 95 - protein, 103 aa, Triticum aestivum
MARTKHPAVRKTKAPPKKQLGPRPAQRRQETDGAGTSATPRRAGRAAAPGGAQGATQAEETTPVQARHGGTAGDQEVSEVGRLSHPVCTICPSDQGGHRLLLS

SEQ ID NO: 96 - DNA, 4399 bp, Triticum aestivum; identical to SEQ ID NO: 87
  source 1..4399: mol_type = other DNA

SEQ ID NO: 97 - RNA, 492 nt, Triticum aestivum; identical to SEQ ID NO: 88
  source 1..492: mol_type = other RNA

SEQ ID NO: 98 - protein, 163 aa, Triticum aestivum; identical to SEQ ID NO: 89

SEQ ID NO: 99 - DNA, 4401 bp, Triticum aestivum; identical to SEQ ID NO: 81
  source 1..4401: mol_type = other DNA

SEQ ID NO: 100 - RNA, 588 nt, Triticum aestivum; identical to SEQ ID NO: 82
  source 1..588: mol_type = other RNA

SEQ ID NO: 101 - protein, 195 aa, Triticum aestivum; identical to SEQ ID NO: 60

SEQ ID NO: 102 - RNA, 493 nt, Triticum aestivum; identical to SEQ ID NO: 85
  source 1..493: mol_type = other RNA

SEQ ID NO: 103 - protein, 106 aa, Triticum aestivum; identical to SEQ ID NO: 86

SEQ ID NO: 104 - DNA, 4399 bp, Triticum aestivum
  source 1..4399: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacaccgtgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggggcaaactgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 105 - RNA, 492 nt, Triticum aestivum
  source 1..492: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccaggcgagccgggcgggcggcggccccagggggggctcaaggggcaaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 106 - protein, 163 aa, Triticum aestivum
MARTKHPAVRKTKAPPKKQLGPRPAQRRQETDGAGTSATPGEPGGRRPQGGLKGQTGQPKQRKPHRFRPGTVALREIRRYQKSVDFLIPFAPFVRLIKEVTDFFCPEISRWTPQALVAIQEAAEYHLVDVFERANHCAIHAKRVTVMQKDIQLARRIGGRRLW

SEQ ID NO: 107 - RNA, 586 nt, Triticum aestivum
  source 1..586: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccgtgcgcgggaccttcccgggtcgcttccttttcgtttcgtcttgttttctgttttttggtctgacttgctcgtcacctgttcgacggaatgcagaggcgagccgggcgggcggcggccccagggggggctcaaggggcaaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 108 - protein, 60 aa, Triticum aestivum
MARTKHPAVRKTKAPPKKQLGPRPAQRRQETDGAGTSATPCAGPSRVASFSFRLVFCFLV

SEQ ID NO: 109 - RNA, 511 nt, Triticum aestivum
  source 1..511: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccgtgcgcgggaccttcccggaggcgagccgggcgggcggcggccccagggggggctcaaggggcaaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 110 - protein, 112 aa, Triticum aestivum
MARTKHPAVRKTKAPPKKQLGPRPAQRRQETDGAGTSATPCAGPSRRRAGRAAAPGGAQGANWATQAEETTPVQARHGGTAGDQEVSEVGRLSHPVCTICPSDQGGHRLLLS

SEQ ID NO: 111 - DNA, 4401 bp, Triticum aestivum; identical to SEQ ID NO: 58
  source 1..4401: mol_type = other DNA

SEQ ID NO: 112 - RNA, 588 nt, Triticum aestivum; identical to SEQ ID NO: 59
  source 1..588: mol_type = other RNA

SEQ ID NO: 113 - protein, 195 aa, Triticum aestivum; identical to SEQ ID NO: 60

SEQ ID NO: 114 - RNA, 513 nt, Triticum aestivum; identical to SEQ ID NO: 61
  source 1..513: mol_type = other RNA

SEQ ID NO: 115 - protein, 170 aa, Triticum aestivum; identical to SEQ ID NO: 62

SEQ ID NO: 116 - RNA, 493 nt, Triticum aestivum; identical to SEQ ID NO: 85
  source 1..493: mol_type = other RNA

SEQ ID NO: 117 - protein, 106 aa, Triticum aestivum; identical to SEQ ID NO: 86

SEQ ID NO: 118 - DNA, 4400 bp, Triticum aestivum; identical to SEQ ID NO: 90
  source 1..4400: mol_type = other DNA

SEQ ID NO: 119 - RNA, 587 nt, Triticum aestivum; identical to SEQ ID NO: 91
  source 1..587: mol_type = other RNA

SEQ ID NO: 120 - protein, 127 aa, Triticum aestivum; identical to SEQ ID NO: 92

SEQ ID NO: 121 - DNA, 4394 bp, Triticum aestivum
  source 1..4394: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacaccggtagcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggtgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 122 - RNA, 486 nt, Triticum aestivum
  source 1..486: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccgaggcgagccgggcgggcggcggccccagggggggctcaaggtgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 123 - protein, 161 aa, Triticum aestivum
MARTKHPAVRKTKAPPKKQLGPRPAQRRQETDGAGTSATPRRAGRAAAPGGAQGGQPKQRKPHRFRPGTVALREIRRYQKSVDFLIPFAPFVRLIKEVTDFFCPEISRWTPQALVAIQEAAEYHLVDVFERANHCAIHAKRVTVMQKDIQLARRIGGRRLW

SEQ ID NO: 124 - RNA, 581 nt, Triticum aestivum
  source 1..581: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccggtagcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 125 - protein, 125 aa, Triticum aestivum
MARTKHPAVRKTKAPPKKQLGPRPAQRRQETDGAGTSATPVARDLPGSLPFRFVLFSVFWSDLLVTCSTECRGEPGGRRPQGGLKVGNPSRGNHTGSGQARWHCGRSGGIRSRSTFSSRLHHLSV

SEQ ID NO: 126 - RNA, 506 nt, Triticum aestivum
  source 1..506: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccggtagcgcgggaccttcccggaggcgagccgggcgggcggcggccccagggggggctcaaggtgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 127 - protein, 144 aa, Triticum aestivum
MDGTVICPFKYVDEVILCSLLNRDERLGSPAADFRTEEVGDLLDQTDKWCKRDEKVDRLLIPPDLPQCHRAWPEPVWFPLLGLPTLSPPWGRRPPGSPPGRSRATGVADVPAPSVSCRRCAGRGPSCFLGGALVFLTAGCLVRA

SEQ ID NO: 128 - DNA, 4401 bp, Triticum aestivum; identical to SEQ ID NO: 58
  source 1..4401: mol_type = other DNA

SEQ ID NO: 129 - RNA, 513 nt, Triticum aestivum; identical to SEQ ID NO: 61
  source 1..513: mol_type = other RNA

SEQ ID NO: 130 - protein, 170 aa, Triticum aestivum; identical to SEQ ID NO: 62

SEQ ID NO: 131 - DNA, 4392 bp, Triticum aestivum
  source 1..4392: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacaccggttgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 132 - RNA, 579 nt, Triticum aestivum
  source 1..579: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccggttgcgcgggaccttcccgggtcgcttccttttcgtttcgtcttgttttctgttttttggtctgacttgctcgtcacctgttcgacggaatgcagaggcgagccgggcgggcggcggccccagggggggctcaaggggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 133 - protein, 192 aa, Triticum aestivum
MARTKHPAVRKTKAPPKKQLGPRPAQRRQETDGAGTSATPVARDLPGSLPFRFVLFSVFWSDLLVTCSTECRGEPGGRRPQGGLKGQPKQRKPHRFRPGTVALREIRRYQKSVDFLIPFAPFVRLIKEVTDFFCPEISRWTPQALVAIQEAAEYHLVDVFERANHCAIHAKRVTVMQKDIQLARRIGGRRLW

SEQ ID NO: 134 - DNA, 4394 bp, Triticum aestivum; identical to SEQ ID NO: 121
  source 1..4394: mol_type = other DNA

SEQ ID NO: 135 - RNA, 581 nt, Triticum aestivum; identical to SEQ ID NO: 124
  source 1..581: mol_type = other RNA

SEQ ID NO: 136 - protein, 125 aa, Triticum aestivum; identical to SEQ ID NO: 125

SEQ ID NO: 137 - RNA, 486 nt, Triticum aestivum; identical to SEQ ID NO: 122
  source 1..486: mol_type = other RNA

SEQ ID NO: 138 - protein, 138 aa, Triticum aestivum
MDGTVICPFKYVDEVILCSLLNRDERLGSPAADFRTEEVGDLLDQTDKWCKRDEKVDRLLIPPDLPQCHRAWPEPVWFPLLGLPTLSPPWGRRPPGSPRCRRRARAICLLPPLRGAGPELLLGRRLGLPDGRVLGAGH

SEQ ID NO: 139 - DNA, 4400 bp, Triticum aestivum; identical to SEQ ID NO: 90
  source 1..4400: mol_type = other DNA

SEQ ID NO: 140 - RNA, 512 nt, Triticum aestivum
  source 1..512: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccggttgcgcgggaccttcccggaggcgagccgggcgggcggcggccccagggggggctcaaggggcaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 141 - protein, 102 aa, Triticum aestivum
MARTKHPAVRKTKAPPKKQLGPRPAQRRQETDGAGTSATPVARDLPGGEPGGRRPQGGLKGQLGNPSRGNHTGSGQARWHCGRSGGIRSRSTFSSRLHHLSV

SEQ ID NO: 142 - RNA, 587 nt, Triticum aestivum; identical to SEQ ID NO: 91
  source 1..587: mol_type = other RNA

SEQ ID NO: 143 - protein, 127 aa, Triticum aestivum; identical to SEQ ID NO: 92

SEQ ID NO: 144 - DNA, 4392 bp, Triticum aestivum; identical to SEQ ID NO: 131
  source 1..4392: mol_type = other DNA

SEQ ID NO: 145 - RNA, 504 nt, Triticum aestivum
  source 1..504: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccggttgcgcgggaccttcccggaggcgagccgggcgggcggcggccccagggggggctcaaggggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 146 - protein, 167 aa, Triticum aestivum
MARTKHPAVRKTKAPPKKQLGPRPAQRRQETDGAGTSATPVARDLPGGEPGGRRPQGGLKGQPKQRKPHRFRPGTVALREIRRYQKSVDFLIPFAPFVRLIKEVTDFFCPEISRWTPQALVAIQEAAEYHLVDVFERANHCAIHAKRVTVMQKDIQLARRIGGRRLW

SEQ ID NO: 147 - DNA, 4399 bp, Triticum aestivum; identical to SEQ ID NO: 87
  source 1..4399: mol_type = other DNA

SEQ ID NO: 148 - RNA, 492 nt, Triticum aestivum; identical to SEQ ID NO: 88
  source 1..492: mol_type = other RNA

SEQ ID NO: 149 - protein, 163 aa, Triticum aestivum; identical to SEQ ID NO: 89

SEQ ID NO: 150 - DNA, 4399 bp, Triticum aestivum; identical to SEQ ID NO: 87
  source 1..4399: mol_type = other DNA

SEQ ID NO: 151 - RNA, 492 nt, Triticum aestivum; identical to SEQ ID NO: 88
  source 1..492: mol_type = other RNA

SEQ ID NO: 152 - protein, 163 aa, Triticum aestivum; identical to SEQ ID NO: 89

SEQ ID NO: 153 - DNA, 4395 bp, Triticum aestivum
  source 1..4395: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacacgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggggcaactgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 154 - RNA, 582 nt, Triticum aestivum
  source 1..582: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacacgcgcgggaccttcccgggtcgcttccttttcgtttcgtcttgttttctgttttttggtctgacttgctcgtcacctgttcgacggaatgcagaggcgagccgggcgggcggcggccccagggggggctcaaggggcaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 155 - protein, 59 aa, Triticum aestivum; identical to SEQ ID NO: 43

SEQ ID NO: 156 - DNA, 4390 bp, Triticum aestivum
  source 1..4390: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacacgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtggtgactaactgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 157 - RNA, 559 nt, Triticum aestivum
  source 1..559: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacacgcgcgggaccttcccgggtcgcttccttttcgtttcgtcttgttttctgttttttggtctgacttgctcgtcacctgttcgacggaatgcagaggcgagccgggcgggcggcggccccagggggggctcaagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 158 - protein, 59 aa, Triticum aestivum; identical to SEQ ID NO: 43

SEQ ID NO: 159 - DNA, 4397 bp, Triticum aestivum; identical to SEQ ID NO: 75
  source 1..4397: mol_type = other DNA

SEQ ID NO: 160 - RNA, 584 nt, Triticum aestivum; identical to SEQ ID NO: 76
  source 1..584: mol_type = other RNA

SEQ ID NO: 161 - protein, 126 aa, Triticum aestivum; identical to SEQ ID NO: 77

SEQ ID NO: 162 - DNA, 4399 bp, Triticum aestivum
  source 1..4399: mol_type = other DNA
atggcccgcaccaagcacccggccgtcaggaagaccaaggcgccgcccaagaagcagctcgggccccgccccgcgcagcggcggcaggagacaggtcagcccttccctccttcctccccccgtctcccgcctctcgagcgaacgccgtcgccatttcgtcgcgagagatggacggacggacgccccacgcatgacgctaatgtgctcttcctctccctttgcagatggcgcgggcacgtcggcgacaccgtgcgcgggaccttcccgggtcgcttccttttcgtttcgtcttgttttctgttttttggtctgacttgctcgtcacctgttcgacggaatgcagaggcgagccgggcgggcggcggccccagggggggctcaaggtgcggccttctttgcgcttttcggttttccgccgcgtgtttagggccatttccgtcttgtttgggggtgcgcggggcgggggcttgttttttttcctccccccttcgttgttgcgcacattgctcgggaatgctgccaggagcggttgcggttcttctttgacccttcgggagggctggatcggcagtttcttcgcttccttgctccagattttagttcatcttgtaccagtacagtagcaagatgatggatgggggcttgttttttctttctccttccttgttccggacatttctctggagcgacttttatgcatttcccagtattgtcctttgtccttagagggtagtggatcggcagttttcttcgcctcattggtccagattttagtttatcttgtacggtaccaagatgatggatgtggcacaaagttctcagtttgggggttgcgctcttccgggcagttgttattttggtctgtgatgactaactcgtatctattcttgtgggagcaggggctaactgggcaacccaagcagaggaaaccacaccggttcaggccaggcacggtggcactgcgggagatcaggaggtatcagaagtcggtcgactttctcatcccgtttgcaccatttgtccgtctggtgggtacctctgtctgtcatatcctctcgctctctctacaaacgatctgcagtgcagagtgtaattggaatattttgttcctgacaaatttgcagatcaaggaggtcaccgacttcttctgtcctgaaatcagccgctggactccccaagcgctcgtcgcgattcaagaggtcagtgctaaaacctggcatgtactattagatctgatggtttgattagagtactacaatgcagatgaattcaatatccgaaaaccatgaactgtggggtagatacatgtatcgccttaattcatggtttctgaatgctctgctattaattcagtttgatatatttatttagcagcatggtattgttttggtggctggtaaatcaaaactgaaatgtgattacgagcaaaacggtatcgattgtcgatcctgtgtgtttttgtgcacatctggttgtttggtcaagatgtgtttgtgcacatcttgcaacatgatcctgcccacacactcaaaactgactattggttaggttccatttgtcttatggaatttagggtgtaactgagaggtgagcaagtggtagtaacgttcaattttgattcaggatgaggatattgtgatccagaaaattgcatgttatggttatgtgtccaaacgccaaatgattatgtctatatccagtactttagaaccagtacaacaacaaaaagtactttagaaccagtcaagtttattgtgcatttatacaagagtgttgtttgcacaatagacttgctttagtcgtctcttgccagaaatgccttcttctgcacaacgagcaaaaataacataaagttgactatactcagtgtggcctaagaaatgagttgtactttttagtcactggcctgtgtatttgctttgaattgacacataattcccttttcctttccctctgcaggctgcagagtatcacctcgtcgacgtatttgaaagggcaaatcactgtgccatccatgcaaagcgtgttaccgtcagtaagttctcactgaatgaaaactccctttcttttacaatattgcgcagaaggaaacatgccagttatgaaagagtttcaattacaggatcacctttgctttcatttgatgtgatatctagttttgatgttgtttcaaggttcaagaattctaatgataaatgataggatccacaattgttatatctctgcagctcctcgtatctgttgtccacgaacaaacatatcaaacaattcattaaaaagatgaagaagtcaaacaaacagtatatgtgcactgcatattagatatcaaacctggttactatacgactgatctggcctgacccccgcgtcgcctctccctggcggcacggggggaaccactccggcgccgccaccttccctcctccctccaccccccacctcgccgccgcctgaggagttcgccggcgaagcccggtctggctccagggagggtggcggcggggcatctctctgcgaggcgtgagggcgcatctcgtgcgcgggcgcggcgagcttgggcgggatcgcgggcgcggctctggctcgggctggtgaggctccggcggtccgaggggtgtcgcggcgcggcgggggtctcgctccggcgcgggcggtccgagggcgcgcgggatctggcgctccagcaggggcgccgtcaaggggagccgggcagggaagctcgtcaggcgggctcgtcaggcaggcgtgacgcggggcggcggccgcgggctggaagccatggcgttttggccatggtgtcgtgtgcgctcgcttctcattcgcagcttgaggtgctgctgctgagggtggtgcgcggtgaagcttggtggtcggcgttggagagtgcaaggcgcaacaggaatggctccaatgatctccacgcttctgggtggatcagatctgcggccctccataggggtgtgttccgggcgaaagccttgacccgactttgtcggtgccgtcgacggcggcgctttcgggcgtcgtttccctccttggaggcgtcgttgtggaactcatcttcttctatgtggggctcgggctctccgggtgaaaacctaagctccagattttccggagcgggcgatggcggcgtcttcgtcgtttccctcttgggggcgttgcttgggagagttagcttgtgcttggtgcgtttggttttctcctacgtcgggtttggtggatgccggggcagcggccccggacggctgatgaacgccgaggcggcggcctcggaaagtgatgcgcggtgcggctccatggggcggggcggtggctcggccttcacttgggtggcaatcttgggccacttgggtggcaggcttgtcggtccggtcgacgcgttccagagggggcggtctgactttgcgtcggggcggcggccccggatgtggtgcgacgttcgtggtctgcgagcggttgccttgagcagcgtgggctgcgggcagctgggtcgcgcggcgttgctgctcgagcggagcggtggtacgtcggggcggcggccccggaaggtgatgccggttgattgcgctgggcgtgacggagcggtggatgtcggggcggcggccccgagaaaatcactgtggcgtccagatttctgtggcaacgatgatggtgggagcgatgtcggcgacgcggcaatggttgcgatagtcggctcttctccggcgtgtccacgatattgcctcggtttgtttgttgctgtggagtcgaagctgcggcggcgaggccctgtggtatacgatgactggttccaggtgtcctttcgtcgatcttccgtggcgccagccgtgcctggtttcgttcttccgagttctccgtcagaatcggagctgcgttgtctgtccgcaggtcgacatgttgtcgagaagggtgggctttgccctgtgtgtttcagtctatgcgagtgggctcggcccttgttgttctggtttttgcccggttttccgtaattaactgggcaattctcttctgcttaattaatagatgaggcaatctttgcctccctttcaaaaaaaaacctggttactatagcaggaaattcagggttgattactttatttcttatctgaaggataaacattgtatcaaatcagaattttatttgtaagttacatttttttttacttataaaacttggaaactgttttactgtgacaaatagatgccactagaatcatgatcacatcgtggctgttgctattctaacaaataaatgctcctgaacaaatgggaactatatatgaagatgtatggaccagcatgttcctgttaacctgacctttttcctttttttgctgctgcagtgcaaaaggacatacagcttgcaaggcgtatcggcgggaggaggctttggtga

SEQ ID NO: 163 - RNA, 586 nt, Triticum aestivum
  source 1..586: mol_type = other RNA
atggcccgcaccaagcacccggccgtcaggaagaccaaggcgccgcccaagaagcagctcgggccccgccccgcgcagcggcggcaggagacagatggcgcgggcacgtcggcgacaccgtgcgcgggaccttcccgggtcgcttccttttcgtttcgtcttgttttctgttttttggtctgacttgctcgtcacctgttcgacggaatgcagaggcgagccgggcgggcggcggccccagggggggctcaaggggctaactgggcaacccaagcagaggaaaccacaccggttcaggccaggcacggtggcactgcgggagatcaggaggtatcagaagtcggtcgactttctcatcccgtttgcaccatttgtccgtctgatcaaggaggtcaccgacttcttctgtcctgaaatcagccgctggactccccaagcgctcgtcgcgattcaagaggctgcagagtatcacctcgtcgacgtatttgaaagggcaaatcactgtgccatccatgcaaagcgtgttaccgtcatgcaaaaggacatacagcttgcaaggcgtatcggcgggaggaggctttggtga

SEQ ID NO: 164 - protein, 60 aa, Triticum aestivum; identical to SEQ ID NO: 108

SEQ ID NO: 165 - DNA, 4399 bp, Triticum aestivum; identical to SEQ ID NO: 87
  source 1..4399: mol_type = genomic DNA

SEQ ID NO: 166 - RNA, 492 nt, Triticum aestivum; identical to SEQ ID NO: 88
  source 1..492: mol_type = genomic RNA

SEQ ID NO: 167 - protein, 163 aa, Triticum aestivum; identical to SEQ ID NO: 89